(19) United States
(12) Patent Application Publication
(10) Pub. No.: US 2026/0022380 A1
(43) Pub. Date: Jan. 22, 2026

(54) GENETIC MODULATORS OF KRAS PROTEIN EXPRESSION AND THEIR USES

(71) Applicant: Northwestern University, Evanston, IL (US)

(72) Inventors: Shana Olwyn Kelley, Evanston, IL (US); Ranjit Singh, Evanston, IL (US); Xi Yue Hu, Evanston, IL (US)

(73) Assignee: Northwestern University, Evanston, IL (US)

(21) Appl. No.: 19/232,130

(22) Filed: Jun. 9, 2025

Related U.S. Application Data
(60) Provisional application No. 63/658,416, filed on Jun. 10, 2024.

(51) Int. Cl. C12N 15/113; A61P 35/00

(52) U.S. Cl. CPC C12N 15/1137; A61P 35/00; C12Y 203/01199; C12N 2310/14; C12N 2310/531; C12N 2320/32

(57) ABSTRACT

A method of inhibiting growth of cancerous cells involves contacting the cancerous cells with a very long chain fatty acid elongase 6 inhibitor (ELOVL6i). The cancerous cells are KRAS dependent. The ELOVL6i reduces a level of KRAS expressed by the cancerous cells, relative to a level thereof prior to contacting of the ELOVL6i, thereby inhibiting the growth of the cancerous cells.

### CROSS-REFERENCE TO RELATED APPLICATIONS

The present application claims priority to U.S. Provisional Patent Application No. 63/658,416 filed Jun. 10, 2024, which is incorporated herein by reference in its entirety.

### SEQUENCE LISTING

The instant application contains a Sequence Listing which has been submitted electronically in XML format and is hereby incorporated by reference in its entirety. Said XML copy, created on Aug. 19, 2025, is named 121384-0272_SL.xml and is 29,178 bytes in size.

### BACKGROUND

Therapeutics that impede the activity of oncogenic mutants of KRAS offer significant clinical impact. Despite the recent success in targeting the G12C mutant, targeted therapy for most prevalent KRAS mutant variants has lagged far behind. Targeting mutant KRAS directly has been challenging due to its structure that lacks deep binding pockets for small-molecule inhibitors. Collectively, over 90% of the KRAS patient population has a mutant variant selected from G12V, G12D, and G13D. There is a need for improved methods to identify allele-specific therapeutic options for mutant KRAS expressing cancers. This includes the need to identify therapeutic options for all KRAS variants.

### SUMMARY

KRAS is a commonly mutated oncogene and therapeutic targeting of its mutants is critical for the development of new cancer therapies (1-4). KRAS mutations are frequently observed in highly fatal cancers, including pancreatic ductal adenocarcinoma (86%), colorectal cancer (41%), non small-cell lung cancer (32%) (2). Notably, the G12 codon is a mutational hotspot accounting for 89% of the mutant KRAS variants (5). Covalent inhibitors have emerged over the last several years that target G12C KRAS via the thiol moiety on cysteine and trap the protein in its GDP-bound inactive form (6, 7). While the KRAS G12C inhibitors have shown potent target engagement and encouraging results in the clinic (8-11), using this strategy to selectively inhibit protein function is not applicable to other KRAS mutants. The G12V KRAS variant, prevalent in colorectal (30%), pancreatic (35%), and lung (23%) cancer, represents a significant therapeutic challenge with no G12V KRAS therapeutics yet available (2).

As described herein, Applicant used magnetic ranking cytometry platform (termed LEAPFROG for large scale phenotypic functional genomics) in combination with whole-genome CRISPR-editing and phenotypic screening using endogenous KRAS protein expression as the readout to identify allele-selective genetic regulators modulating levels of mutant KRAS protein that can be developed as potent anti-cancer agents for cells addicted to continued expression of mutant KRAS protein.

One embodiments is a method of inhibiting growth of cancerous cells. The method comprises contacting the cancerous cells with a very long chain fatty acid elongase 6 inhibitor (ELOVL6i). The cancerous cells are KRAS dependent. The ELOVL6i reduces a level of KRAS expressed by the cancerous cells, relative to a level thereof prior to contacting of the ELOVL6i, thereby inhibiting the growth of the cancerous cells. In some embodiments, the KRAS may include a variant KRAS having at least one substitution relative to a wildtype human KRAS allele. In some embodiments, the at least one substitution of the variant KRAS may be at a position selected from G10, G12, G13, V14, L19, Q22, D33, A59, G60, Q61, R68, H95, Y96, K117 and A146, relative to the wildtype human KRAS allele. Yet in some embodiments, the at least one substitution of the variant KRAS may be selected from G12C, G12R, G13D, Q61X, R68S, H95X, Y96C, A146T, G12V and G12D, relative to the wildtype human KRAS allele. In some embodiments, the contacting may result in a selective reduction of the level of the variant KRAS, relative to a level of the wildtype human KRAS allele. In some embodiments, the contacting may result in a reduction of a level of KRAS inside the cancerous cell, as compared to a level inside the cancerous cell prior to the contacting. In some embodiments, the ELOVL6i may be present in a drug composition, such as an oral drug composition, i.e. a drug composition formulated for oral delivery.

Yet another embodiment is a method of treating cancer that expresses a variant KRAS having at least one substitution relative to a wildtype human KRAS allele in a subject, such as a human being, in need thereof. The method comprises administering to the subject a drug composition that comprises a very long chain fatty acid elongase 6 inhibitor (ELOVL6i). The administering reduces a level of the variant KRAS in the subject, relative to a level of the variant KRAS prior to the administering, thereby treating the cancer that expresses the variant KRAS in the subject. In some embodiments, the method may further comprise detecting the presence of the variant KRAS in the subject prior to the administering. In some embodiments, the at least one substitution of the variant KRAS may be at a position selected from G10, G12, G13, V14, L19, Q22, D33, A59, G60, Q61, R68, H95, Y96, K117 and A146, relative to the wildtype human KRAS allele. Yet in some embodiments, the at least one substitution of the variant KRAS may be selected from G12C, G12R, G13D, Q61X, R68S, H95X, Y96C, A146T, G12V and G12D, relative to the wildtype human KRAS allele. In some embodiments, the at least one substitution of the variant KRAS may be a G12V or G12D substitution, relative to the wildtype human KRAS allele. In some embodiments, the administering may result in a selective reduction of the level of the variant KRAS in the subject, relative to a level of the wildtype human KRAS allele. In some embodiments, the administering may result in a reduced level of ERK phosphorylation and ki-67, relative to a level of ERK phosphorylation and ki-67 prior to the administering. In some embodiments, the administering may result in a reduced level of AKT phosphorylation, relative to a level thereof prior to the administering. In some embodiments, the administering may result in a reduced level of GTP-bound KRAS, relative to a level of GTP-bound KRAS prior to the administering. In some embodiments, the administering may result in a reduced level of very long chain fatty acids in a plasma membrane of a cancer cell of the cancer that expresses the variant KRAS, relative to a level of very long chain fatty acids in the plasma membrane of the cancer cell of cancer that expresses the variant KRAS prior to the administering, thereby reducing anchoring of KRAS to the plasma membrane of the cancer cell of the cancer that expresses the variant KRAS. In some embodiments, the ELOVL6i may be a small molecule having less than 1000 Da molecular weight. In some embodiments, the administering may be performed orally. In some embodiments, the method may reduce an expression level of a very long chain fatty acid elongase 6 (ELOVL6) relative to a level thereof prior to the administration of ELOVL6i. In some embodiments, the ELOVL6i may be a short interfering RNA (siRNA) or short hairpin RNA (shRNA). In some embodiments, the cancer may be c-MYC dependent. In some embodiments, the cancer that expresses the variant KRAS may be a solid tumor cancer. In some embodiments, the cancer that expresses the variant KRAS may be colon cancer, lung cancer, skin cancer, bile duct cancer, uterine endometrial carcinoma, testicular germ cell cancer, cervical squamous cell carcinoma, multiple myeloma or pancreatic cancer.

According to another embodiment is a method to identify genetic modulators genes of KRAS protein expression in a KRAS mutant variant, the method including obtaining genome-wide CRISPR edited cells for cancer cell line populations expressing either wild type KRAS or a KRAS mutant variant, using microfluidic cell sorting to sort cells in each population into endogenous KRAS protein levels based subpopulations, selecting low KRAS-expressing cells; and identifying candidate genetic modulator genes in the low KRAS-expressing cells. In this method the low KRAS-expressing cells represent about the bottom 1-25% of KRAS-expressors in each population. Further, the candidate genetic modulators genes are enriched in the KRAS mutant variant and not enriched in the wild type KRAS and, the candidate genetic modulator gene encodes a protein associated with KRAS functional regulation and/or production in the KRAS mutant variant but not in the wild-type KRAS cell population. In some embodiments the KRAS mutant is selected from G12C, G12R, G13D, Q61X, A146T, G12V and G12D. In some embodiments the KRAS mutant variant cell population is from a colon cancer, a lung cancer, or a pancreatic cancer sample. In some embodiments the KRAS mutant variant cell population is a cell line selected from SW1573, Mia-paca2, Capan-2, Panc 02.03, HCT116, SW480, PSN-1, Panc 03.27, SW403, NCI-H441, GP2D, LS180, COR-L23, LS1034, Capan-1, SK-LU-1, SW1990, NCI-H1944, LS513, Panc 04.03, NCI-H647, NCI-H2444, Calu-6, A427, HUP-T4, CFPAC-1, SK-CO-1, Aspc-1, NCI-H727, and HPAC

According to yet another embodiment is a genetic modulator identified by the methods described herein, wherein the genetic modulator is identified as an allele-selective KRAS modulator, wherein the genetic modulator is associated with controlling KRAS levels in at least one KRAS mutant variant. In some aspects the genetic modulator is ELOVL6, NCAPG, SLC9B2, TGM6, SDE2, PHB2, or PGD.

According to yet another embodiment is a method of treatment of cancer in a subject in need thereof, wherein the method includes determining the KRAS mutant variant in the subject in need, and applying an inhibitor of the genetic modulator, the genetic modulator determined by the methods as described herein, where the cancer is dependent or addicted on KRAS, and wherein the inhibitor reduces KRAS levels by the subject in need, thereby inhibiting the cancer. According to some aspects the inhibitor is applied orally. According to some embodiments the cancer is colon cancer, lung cancer, or pancreatic cancer.

According to another embodiment is a method of inhibiting the growth of cancer cells where the method includes determining the KRAS mutant variant in the cell population contacting the cells with an inhibitor of the genetic modulator, the genetic modulator determined by the methods described herein, where the cancer cells are dependent or addicted on continued KRAS expression for cell survival, and where the inhibitor reduces KRAS levels in the cells, thereby inhibiting the growth of the cells. According to some aspects the inhibitor is applied in vitro, in vivo, ex vivo, and/or in situ.

Applicant discovered ELOVL6 as a highly selective genetic regulator of mutant KRAS protein levels. ELOVL6 functional activity attenuation thorough both genetic and chemical perturbation was shown to exhibit potent anti-tumor activity in both in vitro and in vivo mutant KRAS model systems.

### BRIEF DESCRIPTION OF THE FIGURES

FIGS. 1A-1F. Identification of novel allele-selective genetic regulators of KRAS. (FIG. 1A) Schematic overview of the experimental workflow for the genome-wide loss-of-function phenotypic CRISPR screens based on KRAS levels. Tissue-matched colorectal cancer cell lines expressing either WT KRAS (HT-29) or homozygous KRAS G12V mutant (SW480) were transduced with whole-genome lentiviral CRISPR knockout guide-RNA library (TKOv3, MOI 0.3). CRISPR-edited HT29 and SW480 cells were sorted into subpopulations based on endogenous total KRAS protein level per cell utilizing magnetic ranking cytometry and a ultra-throughput microfluidic cell sorting platform. (FIG. 1B) Density map of individual datapoints overlayed using a colored scale as indicated. Top-ranked genes enriched only in the mutant KRAS expressing cells (red circle) or the WT KRAS expressing cells (blue circle) are highlighted. The genes enriched in both cell types are expected to fall along the dashed line. (FIG. 1C) Pathway enrichment analysis specific to mutant KRAS G12V expressing cells was calculated by Fisher's exact test. (FIGS. 1D-1F) Flow cytometry analysis of parental and CRISPR-edited ELOVL6 knockout cells showing the total KRAS protein level per cell determined by mean fluorescence intensity in (FIG. 1D) WT KRAS expressing HT29 cell line, (FIG. 1E) homozygous G12V mutant KRAS expressing SW480 cell line, and (FIG. 1F) heterozygous G12V mutant KRAS expressing NCI-H441 cell line. Figure discloses SEQ ID NOS 28-31 and 31-32, respectively, in order of appearance.

FIGS. 2A-2K. Loss of ELOVL6 functional activity leads to selective degradation of mutant KRAS protein. (FIGS. 2A-2B) Western blot based total KRAS protein expression assessment in unmodified parental or CRISPR-edited (FIG. 2A) WT KRAS expressing HT29 or (FIG. 2B) homozygous KRAS G12V mutant SW480 cells. Data shown as percent of normalized protein expression of control band density. GAPDH was used as an internal total protein loading control. (FIG. 2C) Dual-antibody based allele-selective KRAS protein detection assay. Cell lysates from wild-type, heterozygous or homozygous mutant KRAS expressing cells were resolved using SDS-PAGE. Immunoblots were sequentially probed with either total-KRAS protein-specific or mutant KRAS G12V-selective antibodies to quantify both total and G12V KRAS mutant protein degradation in unmodified and CRISPR-edited heterozygous G12V KRAS NCI-H441 cell line. The HT29 and SW480 cell line pair were used to demonstrate the selectivity of anti-KRAS G12V antibody. (FIG. 2D) HT29 mCherry-KRAS expressing cells were generated by lentiviral transduction of mCherry-G12V mutant or mCherry-WT KRAS plasmids. Impact of loss of ELOVL6 functional activity on mutant KRAS protein level was evaluated in CRISPR-edited ELOVL6 KO cells. (FIGS. 2E-2F) Bar-graphs summarizing the RT-PCR based total mRNA expression analysis from unmodified parental or CRISPR-edited (FIG. 2E) homozygous or (FIG. 2F) heterozygous G12V KRAS mutant cells. P-values were calculated by two-tailed unpaired T-test. (FIG. 2G) KRAS protein expression assessed after ELOVL6i treatment at different concentrations in SW480 cells. (FIGS. 2H-2I) Representative fluorescent micrographs from live cell imaging of the plasma cell membrane (green) and (FIG. 2H) mCherry-G12V KRAS or (FIG. 2I) mCherry-WT KRAS (red) to assess the impact of ELOVL6i treatment. (FIGS. 2J-2K) Fluorescent intensity quantification of KRAS membrane localization upon ELOVL6i treatment. Two-way ANOVA with Tukey's multiple comparisons test to determine statistical significance. *P<0.05, **P<0.01, ***P<0.001, ***P<0.0001, data are represented as mean±s.d.

FIGS. 3A-3G. ELOVL6 functional activity attenuation mitigates aberrant mutant KRAS signaling. (FIGS. 3A-3B) Colony formation and MTT cell proliferation assays assessing ELOVL6 KO in (FIG. 3A) WT KRAS expressing HT29 or (FIG. 3B) G12V KRAS expressing SW480 colorectal cancer cell lines. P-values were calculated by two-tailed unpaired T-test or two-way ANOVA with Tukey's multiple comparisons test for cell growth curves. (FIG. 3C) Immunoblot analysis for ERK-phosphorylation in mutant KRAS G12V cell lines with ELOVL6 KO. (FIG. 3D) Immunoblot for active-KRAS pulldown analysis of HEK293 cells express with mCherry-WT KRAS or mCherry-G12V KRAS after treatment with ELOVL6i. (FIGS. 3E-3G) Immunoblot analysis with active-KRAS pull down assay demonstrating cellular GTP-bound KRAS levels in mutant KRAS cell lines with genetic modulations of ELOVL6, or chemical perturbation based ELOVL6 enzymatic activity inhibition. Data shown as percent of normalized protein expression of control band density. *P<0.05, **P<0.01, ***P<0.001, ****P<0.0001, data are represented as mean ±s.d.

FIGS. 4A-4J. ELOVL6 functional activity attenuation has in vivo anti-tumor activity and pan mutant KRAS targeting therapeutic potential. (FIG. 4A) Schematic overview of ELOVL6 inhibitor treatment in a heterozygous G12V KRAS mutant SW403 xenograft model for evaluation of KRAS-driven tumor growth and oncogenic signaling. (FIG. 4B) Representative tumor size of the vehicle control and ELOVL6i treated group on day 14 and day 35. (FIGS. 4C-4D) Quantification of (FIG. 4C) tumor size and (FIG. 4D) survival rate in the control and treated groups (n=8). Two-way ANOVA with Tukey's multiple comparisons test to determine statistical significance. Log-rank test to determine the p-value for survival curve. (FIG. 4E) IHC staining with anti-KRAS antibody (top panel), anti-phospho-ERK antibody (middle panel), and cell proliferation marker using anti-ki-67 antibody (bottom panel) for comparison of oncogenic signaling and cancer cell proliferation in ELOVL6i treated tumor cells. (FIG. 4F) Summary of IC50 values assessed over a 7-day period of ELOVL6i treatment in twenty-eight KRAS mutant cell lines, evaluating the effect of ELOVL6 inhibition on cell viability. (FIGS. 4G-4I) Representative bar graphs illustrating the efficacy of ELOVL6i in (FIG. 4G) WT KRAS, (FIG. 4H) G12V KRAS, and (FIG. 4I) G12D KRAS cell lines. Statistical significance was determined by two-tailed unpaired T-test. (FIG. 4J) Analysis of cell line sensitivity to ELOVL6i, plotted based on KRAS dependency scores and IC50 values of each cell line. *P<0.05, **P<0.01, ***P<0.001, ****P<0.0001, data are represented as mean ±s.d.

FIGS. 5A-5G. ELOVL6 inhibitor treatment alters the cellular lipid composition. (FIG. 5A) Targeted fatty acyls (FA) analysis shows relative FA abundance of oleic acid and stearic acid in NCI-H727 control and ELOVL6 inhibitor treated cells. (FIGS. 5B-5C) Relative lipid abundance calculated based on molar fractions of (B) glycerolipid and (C) sphingolipids from lipidomic analysis in NCI-H727 control and ELOVL6i treated cells. Statistical significance was determined by two-tailed unpaired T-test. (FIG. 5D) Relative FA abundance of long-chain PUFAs. (FIG. 5E) Heatmap of PtdSer species in NCI-H727 control and ELOVL6i treated cells, shown as log2 fold changes. (Dark blue=increase, light blue=decrease). (FIG. 5F) Schematic illustration depicting the alterations in lipid compositions induced by ELOVL6 inhibition and the consequent effects on G12V KRAS PM localization. (FIG. 5G) Cell viability assessed by adding back exogenous oleic acid or stearic acid during ELOVL6i treatment in NCI-H727 cell line. P-values were calculated by two-tailed unpaired T-test. *P<0.05, **P<0.01, ***P<0.001, ****P<0.0001, data are represented as mean ±s.d

FIGS. 6A-6C. High-throughput microfluidic cell sorting platform based on magnetic ranking cytometry to sort TKOv3 library modified cells. (FIG. 6A) Schematic design of the microfluidic chip. The fluidic channel incorporates X-shapes microfabricated structure forming capture pockets to trap the cells. Cells carrying high magnetic particles acquire a stronger magnetic force, overcoming drag forces and becoming trapped in zone 1, indicating high KRAS protein expression. The outlet connects to a syringe for the collection of cells with low KRAS expression. (FIG. 6B) Representative flow cytometry profile of KRAS protein expression in HT-29 cells after sorting. (FIG. 6C) Cell sorting profiles of the screened cells.

FIGS. 7A-7B. Quality control check for the whole genome wide CRISPR knockout screens. (FIG. 7A) Assessment of total reads per sample. (FIG. 7B) Distribution of Log2 read counts across samples.

FIGS. 8A-8C. sgRNA enrichment analysis in genome-wide screens. (FIGS. 8A-8B) Scatter plot of individual gene enrichment scores (MAGeCK pipeline) for (FIG. 8B) wild-type KRAS (HT-29) and (FIG. 8A) homozygous KRAS G12V mutant expressing cells (SW480). (FIG. 8C) Intersection of the top 500 hits identified in the screens conducted on HT-29 and SW480 cell lines with low KRAS expression.

FIG. 9. Assessment of ELOVL6 therapeutic potential. Upregulation of ELOVL6 expression in cancer tumor tissues across various cancer types. Data extracted from TCGA database.

FIGS. 10A-10H. Generation of ELOVL6 knockout and knockdown using CRISPR and shRNA technology. ELOVL6 mRNA expression assessed by qPCR in (FIGS. 10A-10E) ELOVL6 monoclonal KO cells and (FIGS. 10F-10H) ELOVL6 shRNA KD cells. Statistical significance was determined by two-tailed unpaired T-test. *P<0.05, **P<0.01, ***P<0.001, ****P<0.0001, data are represented as mean ±s.d.

FIGS. 11A-11C. ELOVL6 inhibition result in reduction in mCherry-tagged G12V KRAS protein expression and phosphorylated ERK signaling. (FIG. 11A) Dose-dependent effects of the ELOVL6 inhibitor on KRAS protein expression were assessed using mCherry-tagged G12V mutant KRAS SW480 cells. (FIG. 11B) Assessment of the impact of ELOVL6 KO in HT-29 cells expressing mCherry-tagged G12V mutant KRAS. Low and high G12V KRAS expressing cells have different levels of ELOVL6 KO determined by mRNA expression. (FIG. 11C) Analysis of phosphorylated ERK expression in ELOVL6 KO cells in G12V expressing HT-29 cell line. Statistical significance was determined by two-tailed unpaired T-test. *P<0.05, **P<0.01, ***P<0.001, ****P<0.0001, data are represented as mean ±s.d.

FIGS. 12A-12C. Assessment of KRAS-driven tumor growth with ELOVL6 inhibitor treatment in heterozygous G12V KRAS mutant NCI-H441 lung cancer model.

(FIG. 12A) Representative tumor size of the vehicle control and ELOVL6 inhibitor (300 mg/kg) treated group on day 10 and day20. (FIG. 12B) Tumor growth curves. (FIG. 12C) Survival rate in the control and treated groups (n=8). Two-way ANOVA with Tukey's multiple comparisons test to determine statistical significance. Log-rank test to determine the p-value for survival curve. *P<0.05, **P<0.01, ***P<0.001, ****P<0.0001, data are represented as mean±s.d.

FIGS. 13A-13D. Attenuation of oncogenic signaling and KRAS-driven tumor growth with ELOVL6 inhibitor treatment in heterozygous G12V KRAS mutant SW403 colon cancer model. (FIG. 13A) Effect of ELOVL6 inhibitor treatment on tumor volume and body weight. Two-way ANOVA with Tukey's multiple comparisons test to determine statistical significance. (FIG. 13B) Western blotting analysis of the impact of ELOVL6 inhibitor treatment on KRAS effector signaling pathways in SW403 tumors collected from mice. (FIG. 13C) Flow cytometry analysis of p-ERK level in treated tumor collected after 3-day, 1-week, and 3-week ELOVL6 inhibitor treatment. (FIG. 13D) p-ERK expression level determined relative to vehicle control treated samples. Two-tailed unpaired T-test to determine statistical significance. *P<0.05, **P<0.01, ***P<0.001, ****P<0.0001, data are represented as mean ±s.d.

FIGS. 14A-14H. Cell viability assessed over 7-day period of ELOVL6 inhibitor treatment across human cancer cell lines with different KRAS mutation backgrounds. (FIGS. 14A-14C) Cell viability in G12V mutant KRAS (FIG. 14A) lung cancer, (FIG. 14B) CRC, and (FIG. 14C) pancreatic cell lines. (FIGS. 14D-14F) Cell viability in G12D mutant KRAS (FIG. 14D) lung cancer, (FIG. 14E) CRC, and (FIG. 14F) pancreatic cell lines. (FIG. 14G) Cell viability in other mutant KRAS cell lines including A146T, G12C, G12R, Q61X, and G13D cell lines. (FIG. 14H) Cell viability in WT KRAS cell lines. Data are represented as mean±s.d.

FIG. 15. KRAS dependency scores. Scatter plot of KRAS dependency scores (x-axis) vs. total KRAS expression (y-axis) for over 1000 human cancer lines across 45 tissue-types. Each data point represents a unique human cancer cell line with (orange) or without (grey) KRAS oncogenic mutation. Sensitivity of individual cell line for continued KRAS protein expression for cell survival is indicated by the scale. Minimum cutoff for KRAS dependency (Chronos score below −1.0) is indicated with a dashed red line. Selected set of cell lines are highlighted to demonstrate the potential impact of KRAS mutation status and KRAS expression levels on context-dependent essentially of KRAS. Data for the scatter plot was extracted from the DepMAP web portal. The location of heterozygous KRAS G12V mutant colorectal cancer cell line (SW403) used to generate the mutant KRAS xenograft model system to evaluate in vivo ELOVL6 anti-tumor therapeutic potential is indicated by a black box.

FIGS. 16A-16B. ELOVL6 FA elongation reduced in cells treated with ELOVL6 inhibitor. Targeted FA analysis revealed the relative abundance of oleic acid and stearic acid in ELOVL6 inhibitor treated cells in (FIG. 16A) HT29 and (FIG. 16B) SW480 cell lines. Statistical significance was determined by two-tailed unpaired T-test. *P<0.05, **P<0.01, ***P<0.001, ****P<0.0001, data are represented as mean ±s.d.

FIGS. 17A-17B. Lipid accumulation in ELOVL6 KO cells. SW480 ELOVL6 KO cells are viable with elevated lipid accumulation assessed through both (FIG. 17A) flow cytometry and (FIG. 17B) immunofluorescence imaging.

FIGS. 18A-18C. ELOVL6 inhibitor treatment shifts the cells with cholesterol-rich lipid compositions. (FIG. 18A) Untargeted lipidomic analysis shows relative lipid abundance of TAG is significantly increased across 3 cell lines. (FIGS. 18B-18C) Relative FA abundance of long-chain PUFA significantly increased upon ELOVL6 inhibitor treatment. Relative abundance is determined based on % mol of each lipid species in the total lipids detected. Untreated cells were used as baseline control. P-values were calculated by two-tailed unpaired T-test.*P<0.05, **P<0.01, ***P<0.001, ****P<0.0001, data are represented as mean ±s.d.

FIGS. 19A-19B. ELOVL6 inhibition driven impact on KRAS-dependent cell survival enhanced by exogenous fatty acids. Cell viability assessed by supplement with exogenous oleic acid or stearic acid upon treatment with ELOVL6i treatment in (FIG. 19A) HPAC, and (FIG. 19B) AsPC-1 cell line. Statistical significance was determined by two-tailed unpaired T-test. *P<0.05, **P<0.01, ***P<0.001, ****P<0.0001, data are represented as mean ±s.d.

### DETAILED DESCRIPTION

The following documents, each of which is incorporated by reference in its entirety, may be useful for understanding this disclosure: (1) WO2011103516; (2) U.S. Provisional Patent Application No. 63/658,415 filed June 2024; (3) T. Nagase, J. Med. Chem. 52, 4111-4114 (2009); (4) Takahashi, J Med Chem 2009 52(10):3142-5.

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

In practicing the present technologies, many conventional techniques in molecular biology, protein biochemistry, cell biology, microbiology and recombinant DNA are used. See, e.g., Sambrook and Russell eds. (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Applicant's allele-specific genetic perturbation approach allowed systematic identification of druggable G12V KRAS modulators that could effectively attenuate G12V KRAS protein levels and aberrant oncogenic signaling. This discovery opens possibilities for allele-selective anti-KRAS therapeutic development based on selective clearance of mutant protein. The technology described herein can be applied to any KRAS mutant of interest to identify genetic modulators for specific KRAS mutants.

The oncogene KRAS encodes the protein GTPase KRAS. It has an mRNA sequence according to NCBI NM_001369786.1.

KRAS mutations can commonly occur at glycine at KRAS position 12 (G12), glycine at position 13 (G13), and glutamine at position 61 (Q61). Mutation can also occur at other positions in KRAS. Each position can have multiple variant mutants, making the number of possible KRAS mutant variants high. See Meng et al (2021) Biomedicine and Pharmacotherapy 140:111717 (doi.org/10.1016/j.biopha.2021.111717). With the heterogeneous response of KRAS mutants to therapeutics, it is therefore important to select KRAS mutant variant specific inhibitors.

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the single forms “a”, “an”, and “the” are intended to include the plural forms as well, unless the context clearly indicates otherwise.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used here, the term “about,” when used to modify a numerical value, indicates that deviations of up to 10% above and below the numerical value, including the numerical value, remain within the intended meaning of the recited value. For example, “about 10” should be understood as both “10” and “9-11”.

As used herein, the term “administering” of an agent to a subject includes any route of introducing or delivering the agent to the subject to perform its intended function. Administration can be carried out by any suitable route, including, but not limited to, intravenously, intramuscularly, intraperitoneally, subcutaneously, and other suitable routes as described herein. Administration includes self-administration and the administration by another.

As used herein, the term “combination therapy” refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the term “effective amount” or “therapeutically effective amount” refers to a quantity of an agent sufficient to achieve a beneficial or desired clinical result upon treatment. In the context of therapeutic applications, the amount of a therapeutic agent administered to the subject can depend on the type and severity of the disease or condition and on the characteristics of the individual, such as general health, age, sex, body weight, effective concentration of the therapeutic agent administered, and tolerance to drugs. It can also depend on the degree, severity, and type of disease. The skilled artisan will be able to determine appropriate dosages depending on these and other factors. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art.

As used herein, the term “reduce” or “decrease” means to alter negatively by at least about 5% including, but not limited to, alter negatively by about 5%, by about 10%, by about 25%, by about 30%, by about 50%, by about 75%, or by about 100%.

In certain embodiments, the terms “disease” “disorder” and “condition” are used interchangeably herein, referring to a cancer, a status of being diagnosed with a cancer, or a status of being suspect of having a cancer.

As used herein, a “cancer” is a disease state characterized by the presence in a subject of cells demonstrating abnormal uncontrolled replication and may be used interchangeably with the term “tumor.” “Cell associated with the cancer” refers to those subject cells that demonstrate abnormal uncontrolled replication.

“Cancer”, which is also referred to herein as “tumor”, is a known medically as an uncontrolled proliferation of abnormal cells in a part of the body, benign or malignant. In one embodiment, cancer refers to a malignant neoplasm, a broad group of diseases involving unregulated cell division and growth, and invasion to nearby parts of the body. Non-limiting examples of cancers include carcinomas, sarcomas, leukemia and lymphoma, colon cancer, colorectal cancer, rectal cancer, gastric cancer, esophageal cancer, head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. As used herein, an advanced cancer refers to a cancer that had progressed after receiving one or more of: the first line therapy, the second line therapy, or the third line therapy.

CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with elements including a Cas gene and specifically designed CRISPRs, nucleic acid sequences can be cut and modified at any desired location. Methods of preparing compositions for use in genome editing using the CRISPR/Cas systems are described in detail in US Pub. No. 2016/0340661, US Pub. No. 20160340662, US Pub. No. 2016/0354487, US Pub. No. 2016/0355796, US Pub. No. 20160355797, and WO 2014/018423, which are specifically incorporated by reference herein in their entireties.

Thus, as used herein, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer”, “guide RNA” or “gRNA” in the context of an endogenous CRISPR system), or other sequences and transcripts from a CRISPR locus. One or more tracr mate sequences operably linked to a guide sequence (e.g., direct repeat-spacer-direct repeat) can also be referred to as “pre-crRNA” (pre-CRISPR RNA) before processing or crRNA after processing by a nuclease.

In some embodiments, one or more vectors driving expression of one or more elements of a CRISPR system are introduced into a target cell such that expression of the elements of the CRISPR system direct formation of a CRISPR complex at one or more target sites. While the specifics can be varied in different engineered CRISPR systems, the overall methodology is similar. A practitioner interested in using CRISPR technology to target a DNA sequence can insert a short DNA fragment containing the target sequence into a guide RNA expression plasmid. The sgRNA expression plasmid contains the target sequence (about 20 nucleotides), a form of the tracrRNA sequence (the scaffold) as well as a suitable promoter and necessary elements for proper processing in eukaryotic cells. Such vectors are commercially available (see, for example, Addgene). Many of the systems rely on custom, complementary oligos that are annealed to form a double stranded DNA and then cloned into the sgRNA expression plasmid. Co-expression of the sgRNA and the appropriate Cas enzyme from the same or separate plasmids in transfected cells results in a single or double strand break (depending of the activity of the Cas enzyme) at the desired target site.

### Methods of Determining Genetic Modulators

The genetic modulators disclosed herein are proteins associated with KRAS production. Specifically, candidate genetic modulators are associated with KRAS production in at least one KRAS mutant variant but not in wild-type KRAS expressing cells.

As described herein is a method for determining genes encoding genetic modulators of KRAS. A broader list of candidate genes may be identified by microfluidic cell sorting, and narrowed down to specific genetic modulators using enrichment analysis.

### Methods of Determining Genetic Modulators

According to one embodiment, described herein is a method for identifying genes and/or proteins associated with KRAS production. The methods may be used to identify allele-selective KRAS modulators, which modulate KRAS activity in specific KRAS mutant variants. The method includes obtaining genome-wide loss-of-function phenotypic CRISPR edited cells for wild type and KRAS mutant variant cell populations, sorting the CRISPR-edited cells using microfluidic cell sorting based on endogenous KRAS protein levels, and identifying the genes associated with low-KRAS expressing cells. These genes are candidate genetic regulators.

In one embodiment, the CRISPR screen is a pooled CRISPR screen, wherein the gRNAs are pooled and added to one population of cells. In another embodiment, the CRISPR screen is an arrayed CRISPR screen, wherein a gRNA is added individually to a pool of cells. The specifics of pooled and arrayed CRISPR screens will be known to those skilled in the art. See. e.g. Bock et al (2022) (doi.org/10.1038/s43586-021-00093-4) for additional information on genome wide CRISPR screens.

After CRISPR editing, the method further includes sequencing the CRISPR-edited cells with the lowest levels of endogenous KRAS, as identified by microfluidic cell sorting. This sequencing will identify the genes responsible for KRAS production in the KRAS mutant and wild type cells.

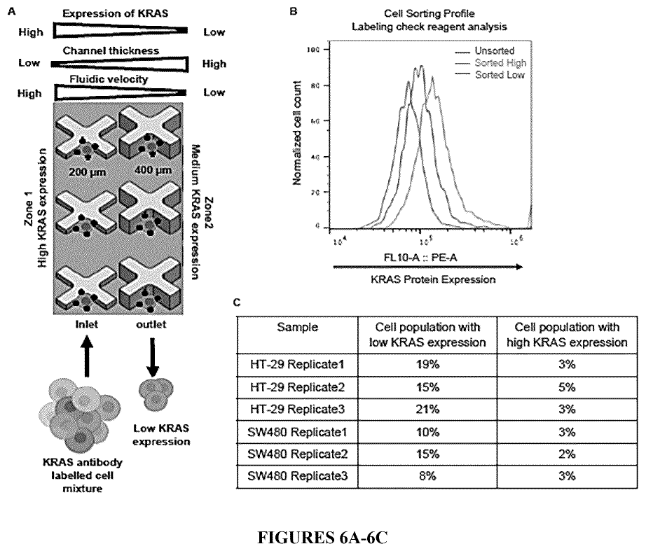

The meaning of “low” and “high” KRAS protein levels can be user defined, as the microfluidic cell sorting parameters can be set by the user. For example, according to the Example described herein, the CRISPR-edited cells were labelled with a biotin-conjugated anti-KRAS antibody, before being labeled with anti-biotin microbeads and sorted magnetically. In this array, higher magnetism indicates higher KRAS. According to the schematic in FIG. 6A, some cells are trapped in pockets of different sizes: high KRAS trapped in zone 1 and medium KRAS trapped in zone 2. Low KRAS cells are collected, not trapped in the cell sorter. The “high,” “medium,” and “low” subpopulations are dependent on the fluidic force (flow rate), capture pocket size and magnetic parameters, and the subpopulations may vary if the parameters are changed. As shown in FIG. 6C, low KRAS cells represented about 5-20% of the total cells in both the wild type and KRAS mutant variant cells, however this number may change depending on the settings of the microfluidic cell sorting.

In the methods described herein, only the cells in the “low” KRAS subpopulation are sequenced. In some embodiments, the candidate genetic modulators are those genes in which the CRISPR mutants are enriched, and the mutants were in the “low” KRAS subpopulation.

In a pooled CRISPR screen, genomic DNA (gDNA) is extracted to prepare next-generation sequencing libraries of PCR amplified gRNAs, sequenced, and enriched gRNAs are identified. To determine enrichment, gRNAs in KRAS mutant variant cells are compared to the gRNAs in the wildtype cells. The enriched gRNAs can be corresponded with a gene. Without wishing to be bound by any particular theory, in the genome-wide CRISPR screens, genes with enrichment in cells with lower endogenous KRAS levels are thought to play a role in KRAS functional regulation and/or production. That is, these genes are thought to play a role in the half-life of the KRAS protein, rate of change of KRAS mRNA production, and/or KRAS protein translation.

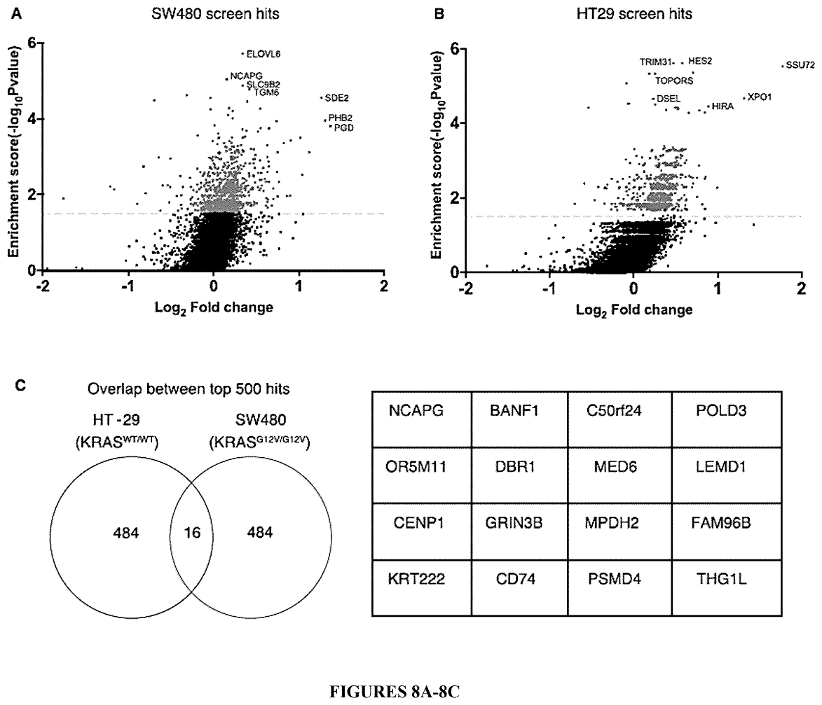

While the definition of “enriched” can be user defined, FIGS. 8A and 8B show that the enriched genes have an enrichment score of higher than approximately 1.5, wherein the enrichment score is represented by the −log₁₀Pvalue. The most highly enriched genes had enrichment scores of >4 in SW480 (KRAS G12V mutant variant) and wild-type KRAS cells.

Alternatively, in an arrayed CRISPR screen, the susceptibility of individual CRISPR alterations is assessed. In an arrayed chemotherapeutic agent CRISPR screen, mutants which have lower KRAS levels likely have a gene knocked out which plays a role in KRAS functional regulation and/or production in the cell.

In one embodiment, the CRISPR screen is performed with the Toronto Knockout V3 (TKOv3) guide RNA library. The TKOv3 library is available for purchase, for example at addgene.org. The TKOv3 library includes guide RNAs (gRNAs) to target 18,053 protein coding-genes in the human genome. In another embodiment, the CRISPR screen may be performed with The Human CRISPR Knockout Pooled (Brunello) sgRNA library. The Brunello sgRNA library is available for purchase, for example at addgene.org. The Brunello sgRNA library includes sgRNAs to make edits (knockdown) over 19,000 genes in the human genome. In other aspects, other sgRNAs are used in the genome-wide CRISPR screen. As used herein, the terms gRNA and sgRNA are used interchangeably.

In some embodiments, the candidate genetic modulators are specific to the wild type or KRAS mutant variant (FIG. 8). In a CRISPR screen with wild type HT-29 cells and KRAS G12V mutant variant SW480 cells, Applicants saw that a majority of biomarker candidate genes were specific to the KRAS variant. Only 16 enriched genes overlapped, comparing the top 500 hits (sequenced gRNA) from each cell population. Therefore, in one embodiment, the candidate genetic modulators genes are enriched in the KRAS mutant variant and not enriched in the wild type KRAS cell population. In one embodiment, as described herein the genetic modulator gene encodes a protein associated with KRAS functional regulation and/or production in a KRAS mutant variant but not in the wild-type KRAS cell population. In some embodiments, the genetic modulator gene is associated with KRAS functional regulation and/or production in more than one KRAS mutant variant.

According to some embodiments, the KRAS mutant variant cell line is from a colon cancer, a lung cancer, or a pancreatic cancer sample. The KRAS mutant variant may be HPAC (G12D), H727 (G12V), or AsPC1 (G12D). G12D and G12V are common KRAS mutant variants.

In some embodiments, the KRAS mutant cancer cell line used in the methods to determine a genetic modulator are other cell lines such as SW1573 (G12C), Mia-paca2 (G12C), Capan-2 (G12-V), Panc 02.03 (G12D), HCT116 (G13D), SW480 (G12V), PSN-1 (G12R), Panc 03.27 (G12V), SW403 (G12V), NCI-H441 (G12V), GP2D (G12D), LS180 (G12D), COR-L23 (G12V), LS1034 (A146T), Capan-1 (G12V), SK-LU-1 (G12D), SW1990 (G12D), NCI-H1944 (G13D), LS513 (G12D), Panc 04.03 (G12D), NCI-H647 (G13D), NCI-H2444 (G12V), Calu-6 (Q61X), A427 (G12D), HUP-T4 (G12V), CFPAC-1 (G12V), SK-CO-1 (G12V), Aspc-1 (G12D), NCI-H727 (G12V), HPAC (G12D). The mutant variant is indicated in parenthesis after each cell name. See FIG. 4F for information about the cell lines.

The cell populations used in the methods herein may have mutant variants including G12V, G12D, G12C, G12R, G13D, Q61X, and A146T. The KRAS mutant variant cell population may be from a cancer including a colon cancer, a lung cancer, or a pancreatic cancer sample.

FIG. 4F shows the tissue origin of multiple KRAS mutant cell lines, and which variant the cell line has.

### Genetic Modulators

As disclosed herein, the methods of genetic modulator identification have identified multiple candidate genetic modulators. In some embodiments, ELOVL6 inhibitors described herein can selectively degrade endogenous RAS mutant variants without impacting wildtype RAS. In some embodiments, ELOVL6 inhibitors described herein can selectively degrade endogenous KRAS mutant variants without impacting wildtype KRAS. In some embodiments, ELOVL6 inhibitors described herein can selectively degrade endogenous HRAS mutant variants without impacting wildtype HRAS. In some embodiments, ELOVL6 inhibitors described herein can selectively degrade endogenous NRAS mutant variants without impacting wildtype NRAS.

A genetic modulator is identified as an allele-selective KRAS modulator, where the genetic modulator is associated with regulating KRAS levels in at least one KRAS mutant variant. In some embodiments, the KRAS mutant variant is selected from G12C, G12R, G13D, Q61X, A146T, G12V and G12D. In some embodiments, the KRAS mutant variant is selected from G12C, G12R, G13D, Q61X, R68S, H95X, Y96C, A146T, G12V and G12D.

In some embodiments, KRAS mutant variants described herein have at least one substitution at positions selected from G10, G12, G13, V14, L19, Q22, D33, A59, G60, Q61, R68, H95, Y96, K117 and A146, relative to the wildtype human KRAS allele. In some embodiments, the at least one substitution of the variant KRAS is selected from G12C, G12R, G13D, Q61X, R68S, H95X, Y96C, A146T, G12V and G12D, relative to the wildtype human KRAS allele. In some embodiments, the at least one substitution of the variant KRAS is a G12V or G12D substitution, relative to the wildtype human KRAS allele.

Some genetic modulators may control KRAS expression of only one KRAS mutant variant, while other genetic modulators may control KRAS levels across multiple KRAS mutant variants.

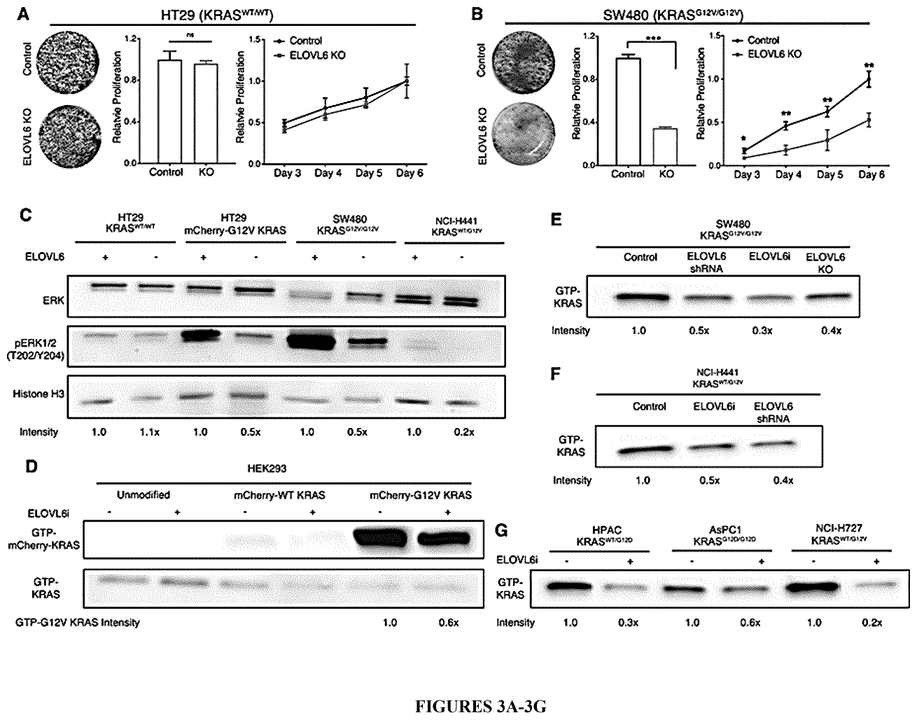

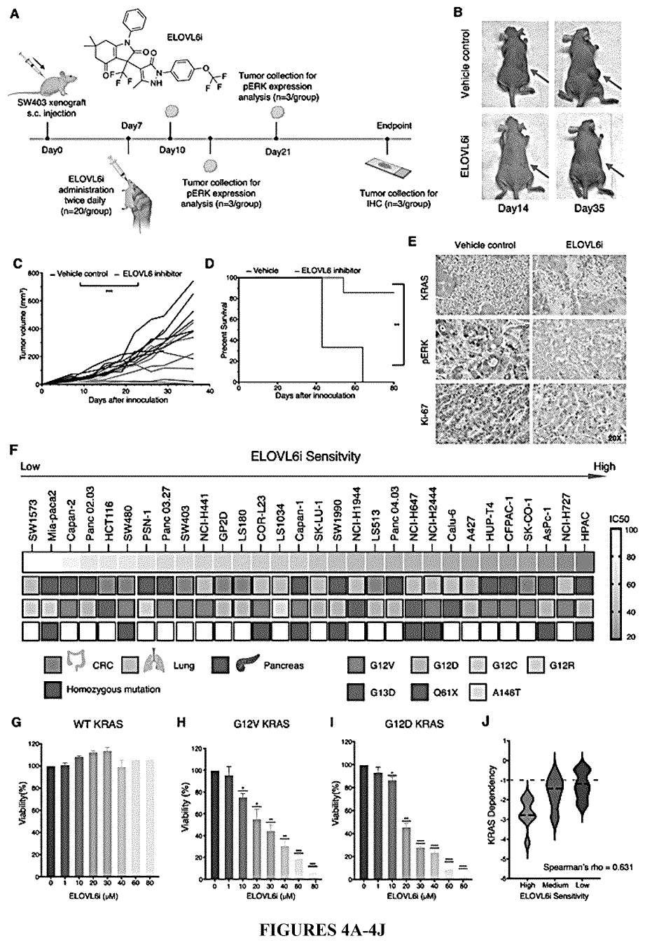

According to some embodiments, the genetic modulator encodes an enzyme. In one embodiment the genetic modulator is ELOVL6. Inhibition of ELOVL6 with a chemical inhibitor significantly reduced KRAS protein levels in KRAS mutant variant cells (SW480 KRAS G12V) but did not significantly reduce KRAS expression in wild type cells (HT29) (FIGS. 3A and 3B). Similar reduction in KRAS was seen in other KRAS mutant cell lines, including cell lines with the G12D variant (FIG. 3G).

Inhibition of the genetic modulator can be done for example, with a chemical compound or by knocking the gene out or mRNA knockdown (FIG. 3E). In one aspect, the chemical inhibitor of ELOVL6 has a structure according to Formula (I).

The genetic modulator may also be selected from NCAPG, SLC9B2, TGM6, SDE2, PHB2, and PGD. These genes, in addition to ELOVL6 were enriched in the CRISPR array of SW480 KRAS G12V mutant cells with low KRAS expression (see FIG. 8A).

ELOVL6 (mRNA NCBI NM_001130721.2) encodes very long chain fatty acid elongase 6; elongation of very long chain fatty acids protein 6.

NCAPG (mRNA NCBI NM_022346.5) encodes condensin complex subunit 3.

SLC9B2 (mRNA NCBI NM_001300754.2) encodes sodium/hydrogen exchanger 9B2.

SDE2 (mRNA NCBI NM_152608.4) encodes splicing regulator SDE2.

PHB2 (mRNA NCBI NM_001144831.2) encodes prohibitin-2.

PGD (mRNA NCBI NM_001304451.2) encodes 6-phosphogluconate dehydrogenase, decarboxylating.

### Methods of Cell Inhibition and Subject Treatment

### Cell Inhibition

According to one embodiment, described herein is a method of inhibiting growth of a cancer cell. In one aspect the cancer selected is colon cancer, pancreatic cancer, and lung cancer. The cancers selected have mutation(s) in the KRAS oncogene. In some aspects the cancer is a tumor.

Methods of inhibition first include determining the KRAS mutant variant in the cancer in the cell population. Methods of determining KRAS mutant variant are known in the art. See e.g. Shakelford et al (2012) Genes and Cancer 3(7-8), doi.org/10.1177/194760191246054. Once the KRAS mutant variant is identified, an inhibitor of the genetic modulator for the mutant variant or composition including the inhibitor may be applied to the cell population Application may be ex vivo, in situ, in vivo or in vitro. The genetic modulator may be identified using the previously described methods.

In some embodiments, ELOVL6 inhibitors described herein are small molecules having less than 1000 Da molecular weight. In some embodiments, ELOVL6 inhibitors described herein are chemical inhibitors. In one embodiment the genetic modulator is ELOVL6 and inhibitor has a structure according to Formula (I).

Alternatively, in some embodiments, ELOVL6 can be inhibited by modifying expression level of ELOVL6. Accordingly, in some embodiments, ELOVL6 can be inhibited by knocking the ELOVL6 gene out or mRNA knockdown. In some embodiments, ELOVL6 inhibitors described herein are short interfering RNAs (siRNAs) or short hairpin RNAs (shRNAs).

In some embodiments, for inhibitors of genetic modulators to be an effective treatment, the cancer should be dependent on continued RAS protein expression for cell survival (context-dependent essentiality and/or synthetic lethality), and the inhibitor is able to induce RAS protein lowering by the subject in need, thereby inhibiting the cancer. In some embodiments, RAS protein comprises NRAS, HRAS, KRAS and variants thereof. In some embodiments, ELOVL6 inhibitors described herein can inhibit functioning of at least one of KRAS, NRAS and HRAS. In some embodiments, ELOVL6 inhibitors described herein can inhibit functioning of at least two of KRAS, NRAS and HRAS. In some embodiments, ELOVL6 inhibitors described herein are PAN-RAS inhibitors that can inhibit functioning of KRAS, NRAS and HRAS. In some embodiments, ELOVL6 inhibitors described herein are selective inhibitors that can inhibit functioning of only one of KRAS, NRAS and HRAS. For example, in some embodiments, the inhibitor of the genetic modulator to be an effective treatment, the cancer should be dependent on continued KRAS protein expression for cell survival (context-dependent essentiality and/or synthetic lethality), and the inhibitor is able to induce KRAS protein lowering by the subject in need, thereby inhibiting the cancer.

### Subject Treatment

According to one embodiment, described herein is a method of treating cancer in a subject. In some embodiments, cancer is a solid tumor cancer. In one aspect, the cancer is selected colon cancer, pancreatic cancer, skin cancer, bile duct cancer, uterine endometrial carcinoma, testicular germ cell cancer, cervical squamous cell carcinoma, multiple myeloma, and lung cancer. In some embodiments, cancer is RAS dependent. In some embodiments, cancer is dependent on KRAS expression. The cancers have mutations in the KRAS oncogene. In some embodiments, the cancer is a tumor.

In some embodiments, cancer is dependent on c-MYC, wherein c-MYC upregulates ELOVL6 expression. Accordingly, in some embodiments, ELOVL6 is overexpressed in cancer subjects. ELOVL6 can regulate ERK phosphorylation, AKT phosphorylation and ki-67. Accordingly, in some embodiments, ELOVL6 overexpression results in upregulation of ERK phosphorylation, AKT phosphorylation and ki-67 expression.

In some embodiments, ELOVL6 inhibitors reduce level of very long chain fatty acids in plasma membrane of cancerous cells. In some embodiments, ELOVL6 inhibitors reduce level of very long chain fatty acids in a plasma membrane of cancerous cells that depend upon RAS protein expression for their survival and proliferation. Accordingly, in some embodiments, ELOVL6 inhibitors induced change in fatty acid composition of plasma membrane negatively affects anchoring ability of RAS protein to the plasma membrane in the cancerous cell. RAS protein has three isoforms: HRAS, NRAS, and KRAS. Accordingly, in some embodiments, ELOVL6 inhibitors can negatively affect anchoring ability of at least one of HRAS (or variants thereof), NRAS (or variants thereof), and KRAS (or variants thereof) to the plasma membrane in cancerous cells. For example, in some embodiments, ELOVL6 inhibitors negatively affects anchoring ability of KRAS or variants thereof to the plasma membrane in cancerous cells. RAS proteins that are anchored to the plasma membrane become active upon binding to GTP. Accordingly, in some embodiments, ELOVL6 inhibitors reduce a level of GTP bound RAS proteins. In some embodiments, ELOVL6 inhibitors can reduce a level of at least one of GTP bound KRAS (or variants thereof), GTP bound HRAS (or variants thereof) and GTP bound NRAS (or variants thereof). For example, in some embodiments, ELOVL6 inhibitors reduce a level of GTP bound KRAS or variants thereof in cancerous cells that depend upon RAS expression for their survival and proliferation.

In some embodiments, ELOVL6 inhibitors alters fatty acid composition of cancerous cells affecting membrane rigidity, permeability and pinocytosis. Accordingly, in some embodiments, ELOVL6 inhibitors induced change in fatty acid composition of plasma membrane can promote uptake of another anticancer agent (e.g., chemotherapeutic agent). In some embodiments, combinations of ELOVL6 inhibitors and another anticancer agent have additive or synergistic anticancer effect. In some embodiments, ELOVL6 inhibitors induced change in fatty acid composition of plasma membrane can increase sensitivity for another anticancer agent. For example, in some embodiments, ELOVL6 inhibitors can promote uptake of paclitaxel in cancerous cells. ELOVL6 inhibitors can improve sensitivity for carboplatin, ibrutinib, lapatinib and afatinib in cancer treatment.

Methods of treatment first include determining the KRAS mutant variant in the cancer in a subject in need. Methods of determining KRAS mutant variant are known in the art. See e.g. Shakelford et al (2012) Genes and Cancer 3(7-8), doi.org/10.1177/194760191246054. Once the KRAS mutant variant is identified, an inhibitor of the genetic modulator for the mutant variant or composition including the inhibitor may be administered to the patient. The genetic modulator may be identified using the previously described methods.

In one embodiment the genetic modulator is ELOVL6 and inhibitor has a structure according to Formula (I).

Administration of the genetic modulator may be accomplished by any method known in the art suitable for the particular type of genetic modulator and formulation selected. Suitable routes of administration include without limitation oral, parenteral (including intramuscular, subcutaneous, intradermal, intravascular, intravenous, intraarterial, intramedullary and intrathecal), intraperitoneal, and topical (including dermal/epicutaneous, transdermal, mucosal, transmucosal, intranasal (e.g., by nasal spray or drop), intraocular (e.g., by eye drop), pulmonary (e.g., by inhalation), buccal, sublingual, rectal and vaginal). Furthermore, the genetic modulator can be formulated for administration by oral inhalation.

For the inhibitor of the genetic modulator to be an effective treatment, the cancer should be dependent on KRAS, and the inhibitor is able to reduce KRAS levels by the subject in need, thereby inhibiting the cancer.

In some embodiments the methods of treatment may increase the overall survival length of the patient. In other embodiments the methods of treatment may reduce the overall size of the tumor (FIGS. 4C-4D).

### Compositions

According to one embodiment, described herein are therapeutic compositions including genetic modulator inhibitors determined by the methods described herein, for example the compound according to Formula (I). The compositions can be used in the methods of treatment and cell inhibition.

In some embodiments, compositions described herein can be used for inhibiting growth of cancerous cells. In some embodiments, the compositions can be used in combination with one or more chemotherapeutic agents. In some embodiments, the compositions can be used for increasing sensitivity of cancerous cells for one or more chemotherapeutic agents. In some embodiments, the compositions can be used for increasing uptake of one of more chemotherapeutic agents. In some embodiments, the compositions can be used for reducing level of RAS or variant thereof in cancerous cells. In some embodiments, the compositions can be used for reducing level of KRAS, HRAS, NRAS or variant thereof in cancerous cells. In some embodiments, the compositions can be used for reducing level of KRAS or variant thereof in cancerous cells. In some embodiments, the compositions can be used for reducing level of ERK phosphorylation in cancerous cells. In some embodiments, the compositions can be used for reducing level of ki-67 in cancerous cells. In some embodiments, the compositions can be used for reducing level of AKT phosphorylation in cancerous cells. In some embodiments, the compositions can be used for reducing level of GTP-bound RAS in cancerous cells. In some embodiments, the compositions can be used for changing fatty acid composition of plasma membrane of cancerous cells. In some embodiments, the compositions can be used for treating cancer. In some embodiments, the compositions can be used for treating solid tumor. In some embodiments, the compositions can be used for treating RAS dependent and/or c-MYC dependent cancer, wherein cancerous cell depend upon RAS and/or c-MYC for their survival and/or proliferation. In some embodiments, the compositions can be used for treating KRAS (or variant thereof) dependent cancer, wherein cancerous cell depend upon RAS and/or c-MYC for their survival and/or proliferation.

In some embodiments, compositions comprising KRAS inhibitors can be used for inhibiting growth of cancerous cells. In some embodiments, compositions comprising KRAS inhibitors can be used in combination with one or more chemotherapeutic agents. In some embodiments, compositions comprising KRAS inhibitors can be used for increasing sensitivity of cancerous cells for one or more chemotherapeutic agents. In some embodiments, compositions comprising KRAS inhibitors can be used for increasing uptake of one of more chemotherapeutic agents. In some embodiments, compositions comprising KRAS inhibitors can be used for reducing level of KRAS or variant thereof in cancerous cells. In some embodiments, compositions comprising KRAS inhibitors can be used for reducing level of ERK phosphorylation in cancerous cells. In some embodiments, compositions comprising KRAS inhibitors can be used for reducing level of ki-67 in cancerous cells. In some embodiments, compositions comprising KRAS inhibitors can be used for reducing level of AKT phosphorylation in cancerous cells. In some embodiments, compositions comprising KRAS inhibitors can be used for reducing level of GTP-bound KRAS in cancerous cells. In some embodiments, compositions comprising KRAS inhibitors can be used for changing fatty acid composition of plasma membrane of cancerous cells. In some embodiments, compositions comprising KRAS inhibitors comprising KRAS inhibitors can be used for treating cancer. In some embodiments, compositions comprising KRAS inhibitors can be used for treating solid tumor. In some embodiments, compositions comprising KRAS inhibitors can be used for treating KRAS dependent and/or c-MYC dependent cancer, wherein cancerous cell depend upon KRAS and/or c-MYC for their survival and/or proliferation. In some embodiments, compositions comprising KRAS inhibitors can be used for treating KRAS (or variant thereof) dependent cancer, wherein cancerous cell depend upon KRAS and/or c-MYC for their survival and/or proliferation.

Such compositions can be formulated and/or administered in dosages and by techniques well known to those skilled in the medical arts taking into consideration such factors as the age, sex, weight, tumor type and stage, condition of the particular patient, and the route of administration.

In some embodiments, the composition formulated for administration has the inhibitor present at a concentration of between 1-200 μM, between 1-100 μM, between 10-100 μM, between 20-100 μM, between 30-100 μM, between 30-100 μM, between 50-100 μM, between 60-100 μM, or between 70-100 μM

The compositions may include pharmaceutical solutions comprising carriers, diluents, excipients, preservatives, and surfactants, as known in the art. Further, the compositions may include preservatives (e.g., anti-microbial or anti-bacterial agents such as benzalkonium chloride). The compositions also may include buffering agents (e.g., in order to maintain the pH of the composition between 6.5 and 7.5).

In some embodiments, compositions described herein are pharmaceutical compositions. The pharmaceutical compositions may be administered therapeutically. In therapeutic applications, the compositions are administered to a patient in an amount sufficient to elicit a therapeutic effect (e.g., a response which cures or at least partially arrests or slows symptoms and/or complications of disease (i.e., a “therapeutically effective dose”).

In some embodiments, compositions are formulated for systemic delivery, such as oral or parenteral delivery. Additional exemplary routes of administration include inhalation, respiration, intranasal, intubation, intrapulmonary instillation, buccal, intrapulmonary, intradermal, topical, dermal, sublingual, subcutaneous, intravascular, intrathecal, intraarticular, intracavity, transdermal, iontophoretic, intraocular, opthalmic, optical, intravenous (i.v.), intramuscular, intraglandular, intraorgan, and/or intralymphatic. In some embodiments, minimally invasive microneedles and/or iontophoresis may be used to administer the composition. In some embodiments, compositions are formulated for site-specific administration, such as by injection into a specific tissue or organ, topical administration (e.g., by patch applied to the target tissue or target organ). In some embodiments, the composition is formulated to be delivered through the blood-brain barrier. By way of example, but not by way of limitation, such methods may include ultrasound treatment with or without concomitant administration of microbubbles, convection enhanced drug delivery, biodegradable wafers that release the drug, peptide-drug conjugates, and nanoparticle-drug coupling to enhance drug penetration.

In some embodiments, the methods include administration of the therapeutic compositions once per day; in some embodiments, the composition may be administered multiple times per day, e.g., at a frequency of one or two times per day, or at a frequency of three or four times per day or more. In some embodiments, the methods include administration of the composition once per week, once per month, or as symptoms dictate.

In some embodiments, the compositions are used in combination with additional therapeutic agents and methods. In some embodiments, the compositions described herein are applied sequentially or consecutively with additional therapeutic agents.

In some embodiments, compositions described herein are formulated for oral administration. In some embodiments, compositions described herein are formulated for intravenous administration.

### Patient Populations

As used herein, the terms “patient,” and “subject” are used interchangeably.

In some embodiments, a subject in need thereof in accordance with the technologies (i.e. methods and compositions) disclosed herein include, but are not limited to, humans and non-human vertebrates. In some embodiments, a subject in need thereof in accordance with the technologies disclosed herein comprise, for example, a mammal. In some embodiments the mammal is a human. In some such embodiments, a mammal includes, for example and without limitation, a household pet (e.g., a dog, a cat, a rabbit, a ferret, a hamster, etc.), a livestock or farm animal (e.g., a cow, a pig, a sheep, a goat, a chicken or another poultry), a horse, a monkey, a laboratory animal (e.g., a mouse, a rat, a rabbit, etc.) and the like. Subjects can also include fish and other aquatic species. In a preferred embodiment, the subject in need thereof in accordance with technologies described herein is a human.

In some aspects, technologies of the present disclose can be utilized in a subject that has pancreatic cancer, lung cancer, colon cancer, or another cancer. A subject that has cancer is a subject that has detectable cancer cells. In some embodiments, a cancer involves one or more tumors. In some aspects, technologies of the present disclose can be utilized in a subject that has colon cancer, lung cancer, skin cancer, bile duct cancer, uterine endometrial carcinoma, testicular germ cell cancer, cervical squamous cell carcinoma, multiple myeloma or pancreatic cancer.

In some aspects, the subject in need thereof in accordance with technologies of the present disclosure has undergone one or more other cancer therapies (e.g., chemotherapy, radiotherapy, immunotherapy). In some embodiments, the subject in need thereof has previously undergone or more other cancer therapies and the subject's cancer has relapsed. In some embodiments, the subject in need thereof has previously undergone one or more other cancer therapies and the subject has developed resistance to the one or more other cancer therapies. In some embodiments, the subject in need thereof is in remission (e.g., partial remission, complete remission). In some embodiments, the subject in need thereof is refractory to one or more other cancer therapies.

In some embodiments, the subject in need thereof is an adult. In some embodiments, the subject in need thereof is a human subject over 18 years of age. In some embodiments, the subject in need thereof is a human subject over 21 years of age. In some embodiments, the subject in need thereof is a human subject over 30 years of age. In some embodiments, the subject in need thereof is a human subject over 65 years of age. In some embodiments, the subject in need thereof is a human subject under 18 years of age. In some embodiments, the subject in need thereof is a human subject under 65 years of age (or between 18 and 65 years of age, between 21 and 65 years of age, or between 30 and 65 years of age). In some embodiments, the subject in need thereof is a pediatric subject (e.g., a human subject under the age of 12).

### EXAMPLES

Therapeutics that impede the activity of oncogenic mutants of KRAS offer significant clinical impact. However, targeting mutant KRAS directly has been challenging due to its structure that lacks deep binding pockets for small-molecule inhibitors. Despite the recent success in targeting the G12C mutant, targeted therapy for another hotspot variant—G12V KRAS—has lagged far behind. Applicant utilized CRISPR/Cas9 based genome-wide knockout screens and leveraged a high-throughput microfluidic cell sorting platform to identify genes that specifically modulate levels of G12V KRAS. The top hit identified from the KRAS screens, a fatty acid elongase (ELOVL6), showed remarkable selectivity in diminishing G12V KRAS mutant protein levels and aberrant oncogenic signaling. The results presented herein reveal that ELOVL6, a druggable enzyme, can be targeted to control the production of lipids that are exploited by mutated forms of KRAS for function and trigger the targeted degradation of the protein.

Given the challenges of directly inhibiting the G12V KRAS variant, Applicant focused instead on identifying genetic regulators of mutated protein levels in cells. By conducting genome-wide CRISPR/Cas9 mediated knockout (KO) screens in wild type (WT) and G12V KRAS cell lines using an immunomagnetic cell sorting approach amenable to comparative parallel screening, Applicant sought to identify modulators that selectively decreased endogenous G12V KRAS protein levels without impacting WT KRAS.

### Results

### Identification of Novel Allele-specific Regulators of G12V KRAS

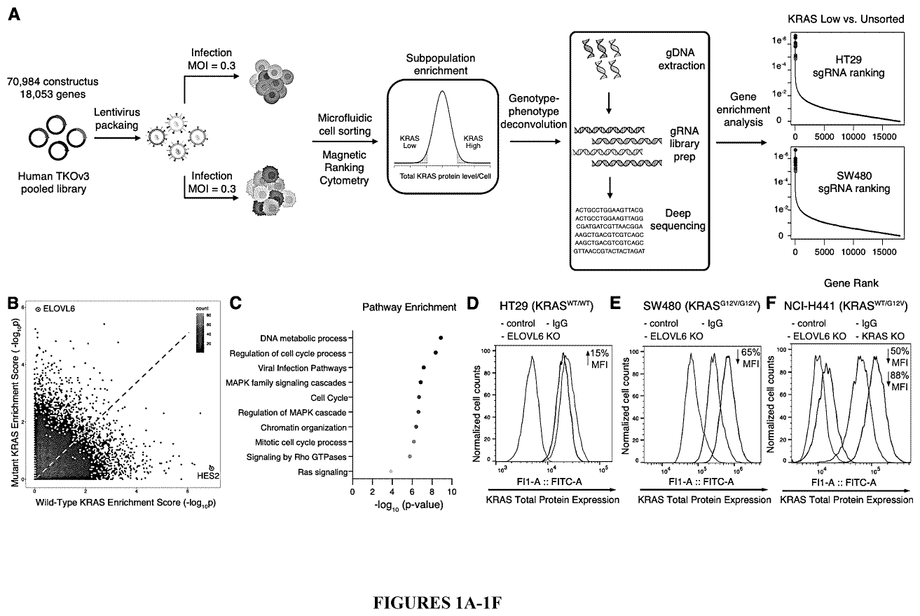

Applicant selected the colorectal cancer cell lines HT29 (KRASWT/WT) and SW480 (KRASG12V/G12V) for genome-wide loss-of-function phenotypic CRISPR/Cas9 screening (FIG. 1A). Cells were transduced with the Toronto Knockout V3 (TKOv3) guide RNA library, containing 70,090 single-guide RNAs (sgRNAs) targeting ˜18,000 human genes (12). To enable parallel screening, Applicant leveraged a high-throughput microfluidic cell sorting platform (FIG. 6A) that utilizes magnetic ranking cytometry (MagRC) (13) to sort CRISPR-edited cells into endogenous KRAS protein levels based subpopulations (FIGS. 6B and 6C). Applicant harvested the low-KRAS expressing cells (10-20% of total cell population) and extracted genomic DNA for next-generation sequencing (NGS) based gRNA enrichment analysis.

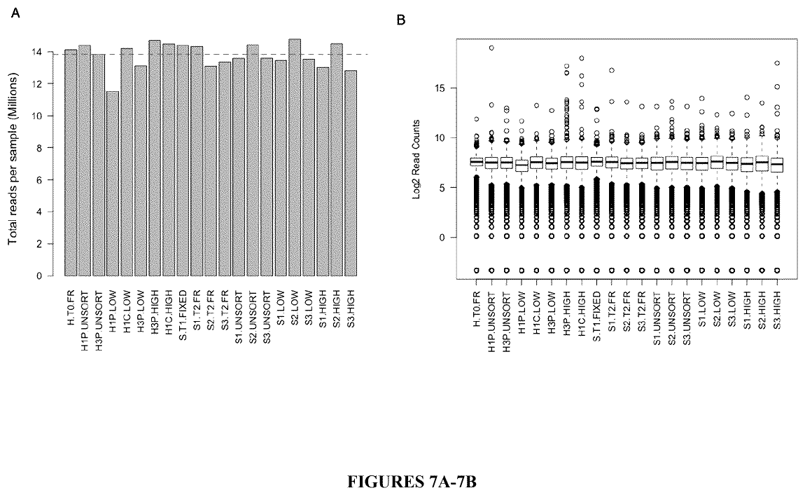

The Model-based Analysis of Genome-wide CRISPR/Cas9 Knockout (MAGeCK) pipeline was used to analyze sequencing data (14). Read count distribution across all samples was analyzed to assess data quality (FIG. 7). A comparison of genes enriched in G12V KRAS or WT KRAS expressing cells (FIGS. 1B and 8) highlighted that distinct sets of modulators could be visualized for each form of KRAS. Pathway enrichment analysis identified biological processes associated with KRAS such as the mitogen-activated protein kinase (MAPK) family signaling cascade (FIG. 1C). Interestingly, gRNA enrichment analysis from SW480 cells (G12V/G12V) identified ELOVL6 (elongation of very long-chain fatty acids family member 6) as the top-ranking gene, with a false discovery rate (FDR) of 0.034.

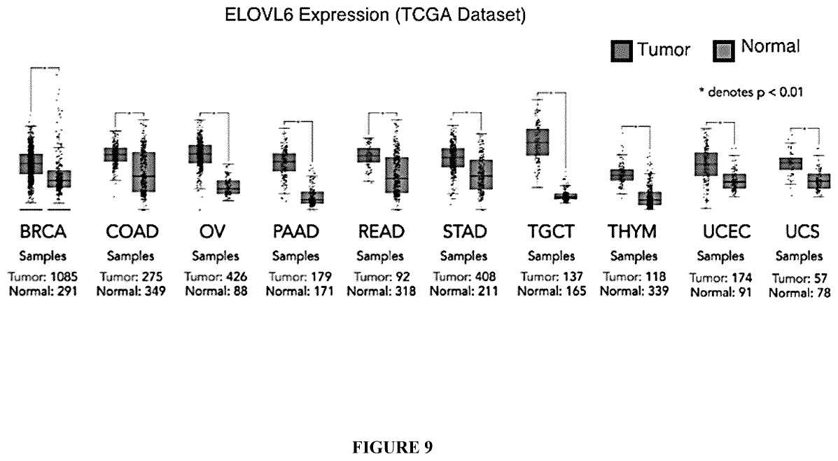

ELOVL6 is a fatty acid elongase (15) with no known link to modulation of KRAS levels. Based on The Cancer Genome Atlas (TCGA) data, ELOVL6 expression is upregulated in many tumor types (FIG. 9). To validate the functional impact of loss of ELOVL6 on KRAS, flow cytometry analyses were conducted on CRISPR-edited ELOVL6 KO cells from SW480 and NCI-H441 (KRASG12V/WT) cell lines. Applicant observed 65% reduction in KRAS mean fluorescence intensity (MFI) in SW480 and 50% reduction in NCI-H441 cells (FIGS. 1E and 1F); no decrease in KRAS protein levels was observed in HT29cells. This confirms the outputs of the phenotypic screens and that ELOVL6 can function as an allele-selective KRAS modulator.

Loss of ELOVL6 functional activity leads to selective degradation of mutant KRAS protein.

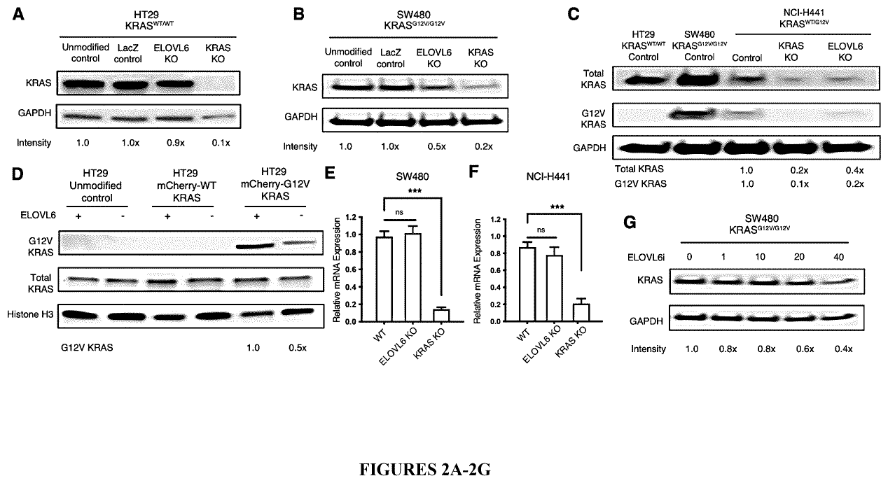

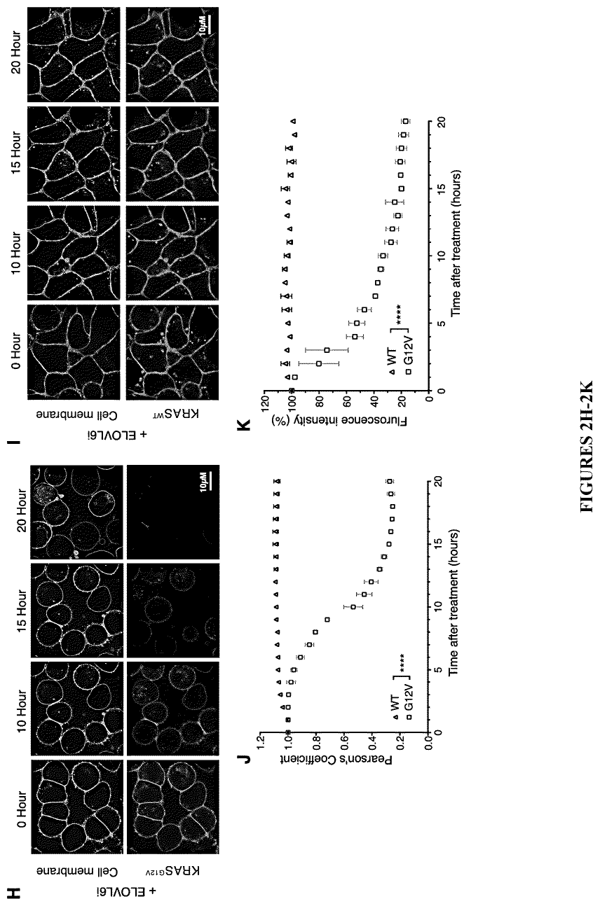

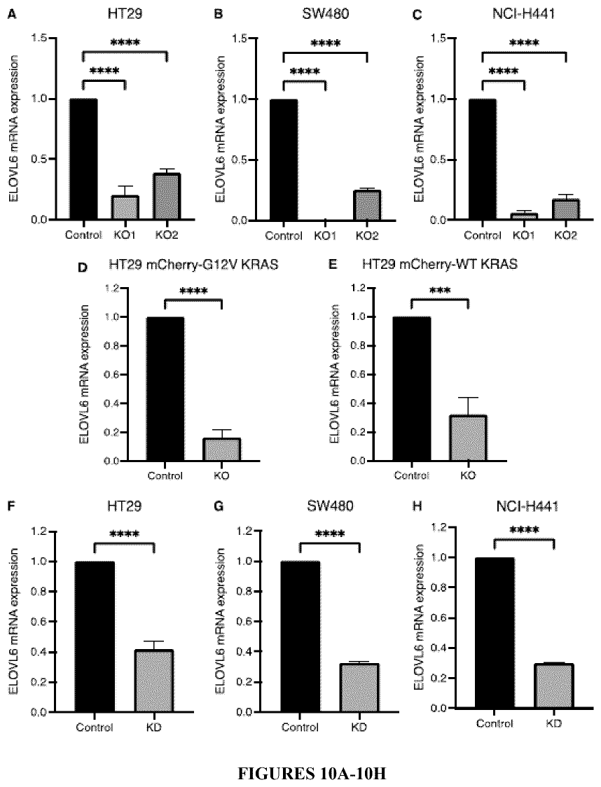

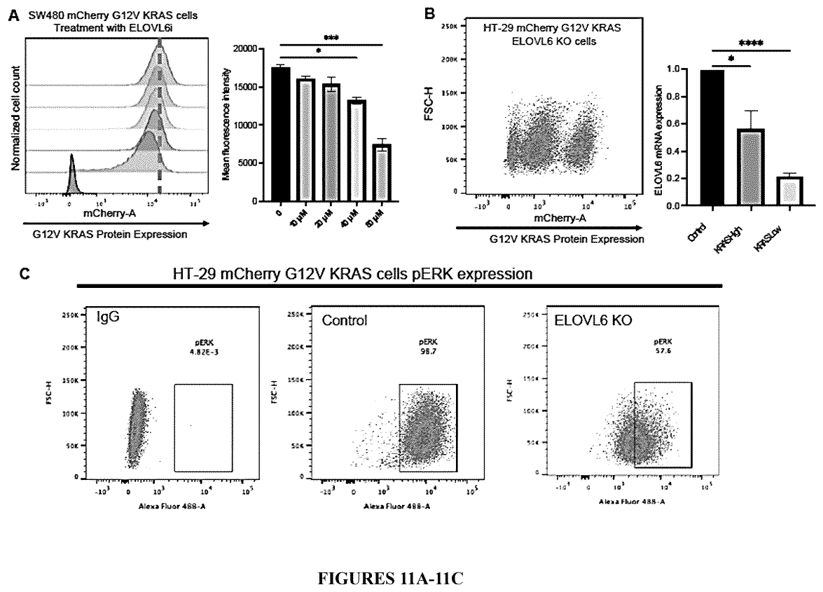

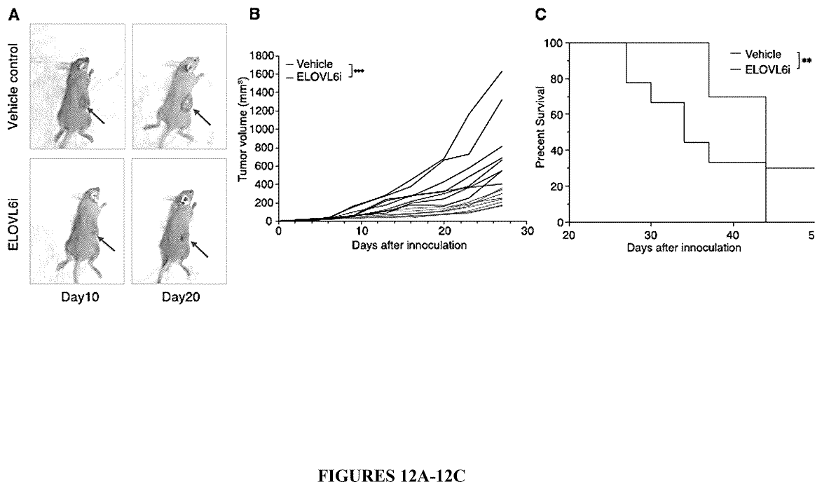

To investigate the functional impact of ELOVL6 on KRAS, monoclonal ELOVL6 KO cells were generated and assessed for ELOVL6 KO efficiency (FIG. 10). A 44% reduction in KRAS protein levels was observed in CRISPR-edited SW480 cells, with minimal reduction in HT29 cells (FIGS. 2A and 2B). To assess the selectivity of ELOVL6 KO towards G12V KRAS, Applicant developed a dual antibody-based KRAS detection assay and used western blotting to assess levels of total and G12V mutant KRAS protein (FIG. 2C). Analyzing NCI-H441 ELOVL6 KO cells with an antibody that captured total KRAS yielded a 60% reduction in total KRAS protein levels. When the analysis was conducted with a mutation-specific anti-G12V KRAS antibody, NCI-H441 ELOVL6 KO cells exhibited 80% reduction in G12V KRAS protein levels. To validate selectivity of ELOVL6 KO, Applicant overexpressed G12V KRAS protein in HT29 cells and observed a 51% reduction in G12V KRAS protein levels when ELOVL6 was knocked out (FIG. 2D). Applicant also examined total KRAS mRNA expression in ELOVL6 KO cells and observed no significant change (FIGS. 2E and 2F). Together, these results suggest that loss of ELOVL6 functional activity does not impact KRAS at the mRNA level but exerts its allele-selective effect at the protein level.

KRAS is only active when properly localized to the cell membrane (2). Since ELOVL6 is required for synthesizing very-long-chain fatty acids (VLCFAs)—essential components of various cellular processes including cell membrane phospholipids (16)—Without wishing to be bound by any particular theory, Applicant hypothesized that loss of ELOVL6 activity could hinder KRAS membrane anchoring and lead to protein degradation. Thus, Applicant examined KRAS protein levels as well as localization at the plasma membrane (PM) as a function of ELOVL6 inhibition. Applicant utilized a potent small molecule ELOVL6 inhibitor (commercially available under the name ELOVL6-IN-2, also referred to herein as ELOVL6i) (17-19) as a tool compound to assess whether ELOVL6 inhibition would alter KRAS levels and membrane anchoring. ELOVL6i has a chemical structure according to formula (I):

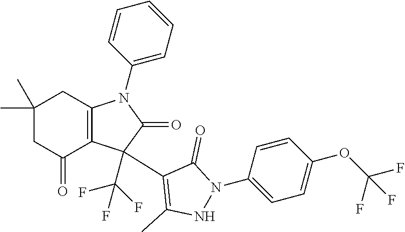 [SMILES: Cc1[nH]n(-c2ccc(OC(F)(F)F)cc2)c(=O)c1C1(C(F)(F)F)C(=O)N(c2ccccc2)C2=C1C(=O)CC(C)(C)C2]

ELOVL6i treatment revealed a concentration-dependent effect on G12V KRAS in SW480 cells (FIG. 2G). To investigate KRAS protein levels and localization without cell death related to KRAS dependency, Applicant generated mCherry-G12V KRAS expressing HT29 cells. Applicant observed dose-dependent effects of ELOVL6i on lowering mCherry-G12V KRAS (FIGS. 11A and 11B). Interestingly, mCherry-G12V KRAS protein levels correlated with ELOVL6 mRNA expression (FIG. 11C). Applicant also conducted live-cell imaging for co-localization analysis utilizing mCherry-KRAS and a PM-specific fluorescent marker to assess KRAS subcellular localization. Significant reductions in both mCherry-G12V KRAS fluorescence intensity and co-localization with cell membrane were observed upon ELOVL6i treatment (FIGS. 2H-2K). These results suggest ELOVL6 functional activity can modulate G12V KRAS PM localization.

### ELOVL6 Functional Activity Attenuation Mitigates Aberrant Mutant KRAS Signaling.

To evaluate the effects of loss of ELOVL6 functional activity, Applicant compared cell proliferation and signaling. Cell proliferation was measured using an MTT assay over 1 week and clonogenic assays over 3 weeks. A significant reduction in colony formation was observed in SW480 ELOVL6 KO cells (FIGS. 3A and 3B) while HT29 cells were unaffected. Findings suggest that ELOVL6 KO induced attenuation of the proliferation rate is G12V KRAS dependent. Applicant also investigated the effects on KRAS effector pathways, specifically the MAPK pathway. ELOVL6 KO caused a signification reduction in ERK phosphorylation, with 48% reduction observed in HT29 cells transduced with mCherry-G12V KRAS protein, a 43% reduction in SW480 cells, and 71% reduction in NCI-H441 cells (FIG. 3C).

Since KRAS mutations disrupt the GDP/GTP exchange cycle and bias mutant KRAS towards a predominantly GTP-bound state (2), Applicant examined the impact of ELOVL6 functional activity on GTP-bound RAS (FIGS. 3D-3G). To examine allele specificity, human embryonic kidney 293 (HEK293) cells were transduced to express G12V KRAS. G12V KRAS expression led to a significant increase in GTP-bound KRAS, while ELOVL6i treatment significantly reduced GTP-bound KRAS in G12V KRAS expressing HEK293 cells (FIG. 3D). Furthermore, ELOVL6 functional activity attenuation through permanent CRISPR/Cas9-mediated KO, transient shRNA-mediated knockdown (KD), or inhibition with ELOVL6i, all consistently reduced cellular GTP-bound KRAS. This effect was observed across multiple cancer cell lines harboring G12V or G12D KRAS (FIGS. 3E-3G). Collectively, the findings elucidate a regulatory role of ELOVL6 on KRAS-dependent survival, aberrantly activated ERK phosphorylation and levels of GTP-bound active KRAS.

### ELOVL6 Functional Activity Attenuation Has In Vivo Anti-tumor Activity and Pan Mutant KRAS Targeting Therapeutic Potential.

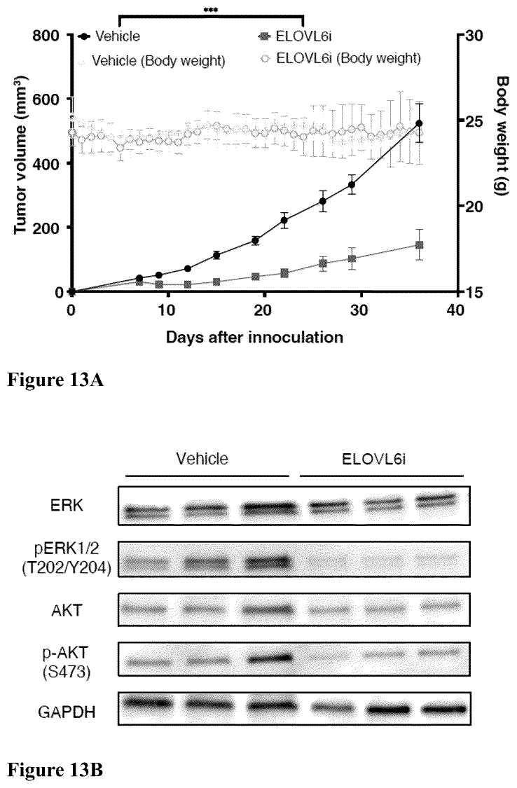

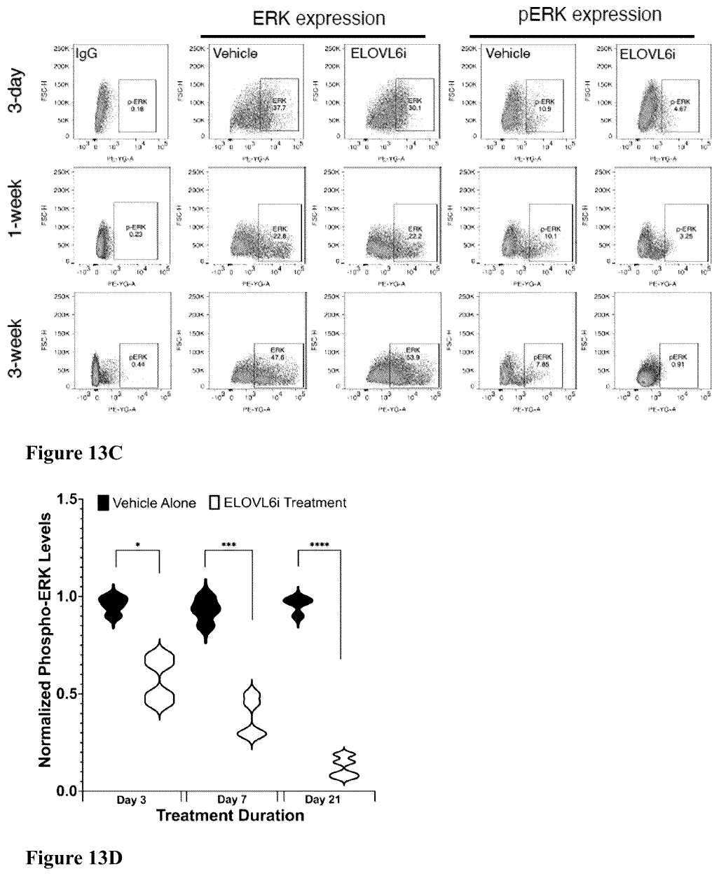

To establish antitumor activity of ELOVL6 inhibition in vivo, Applicant investigated the efficacy of ELOVL6i in G12V KRAS mutant xenograft models (FIGS. 4A and 13). Daily oral administration of ELOVL6i in mice bearing SW403 xenograft was well tolerated (FIG. 13A). ELOVL6i-treated mice exhibited a significant reduction in tumor growth rate and prolonged survival (FIGS. 4B-4D). Applicant also examined KRAS-associated ERK and AKT phosphorylation in tumor tissue. Tumor samples collected from mice treated with ELOVL6i displayed a time-dependent reduction in ERK phosphorylation (FIGS. 13B-13D). Immunohistochemistry-based KRAS, phospho-ERK, and ki-67 proliferation marker analysis in ELOVL6i-treated samples exhibited a significant decrease in the expression of mutant KRAS, ERK phosphorylation, and ki-67 (FIG. 4E). Together, these findings strongly suggest that ELOVL6i treatment produces anti-tumor effects, manifesting as significant reductions in tumor progression and mitigation of mutant KRAS-associated aberrant signaling.

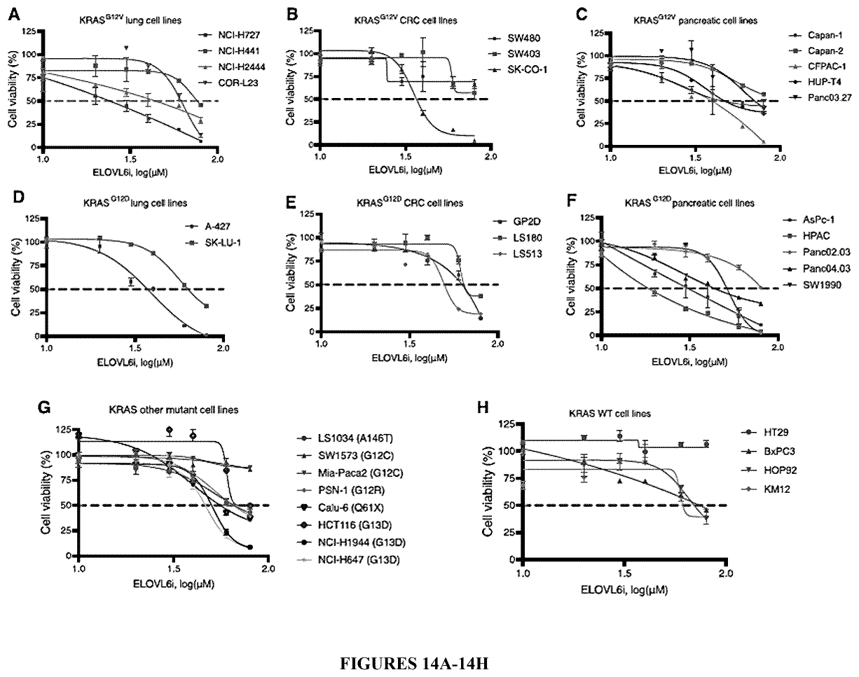

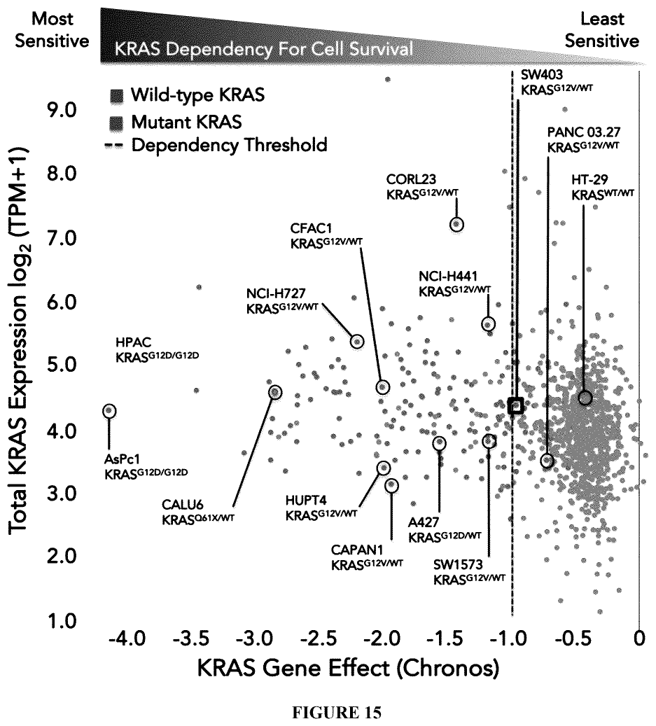

Next, Applicant evaluated the efficacy of ELOVL6i across a panel of human cell lines, originating from colorectal, lung, and pancreatic cancers. This panel included Thirty cell lines harboring KRAS mutations, including G12V (n=12), G12D (n=9), G13D (n=3), G12C (n=3), G12R (n=1), Q61X (n=1), and A146T (n=1). Treatment with the ELOVL6i impacted cell viability across all KRAS variants tested (FIG. 4F). In general, mutant KRAS-expressing cells were more sensitive to ELOVL6i treatment than the WT KRAS expressing cells (FIGS. 4G-4I, and 14). Since mutant KRAS cell lines exhibit survival dependency on continued KRAS expression, Applicant extracted the relevant dependency scores from DepMap (FIG. 15) and found a significant correlation between KRAS dependency scores and ELOVL6i sensitivity (FIG. 4J).

### ELOVL6 Inhibitor Treatment Alters the Cellular Lipid Composition.

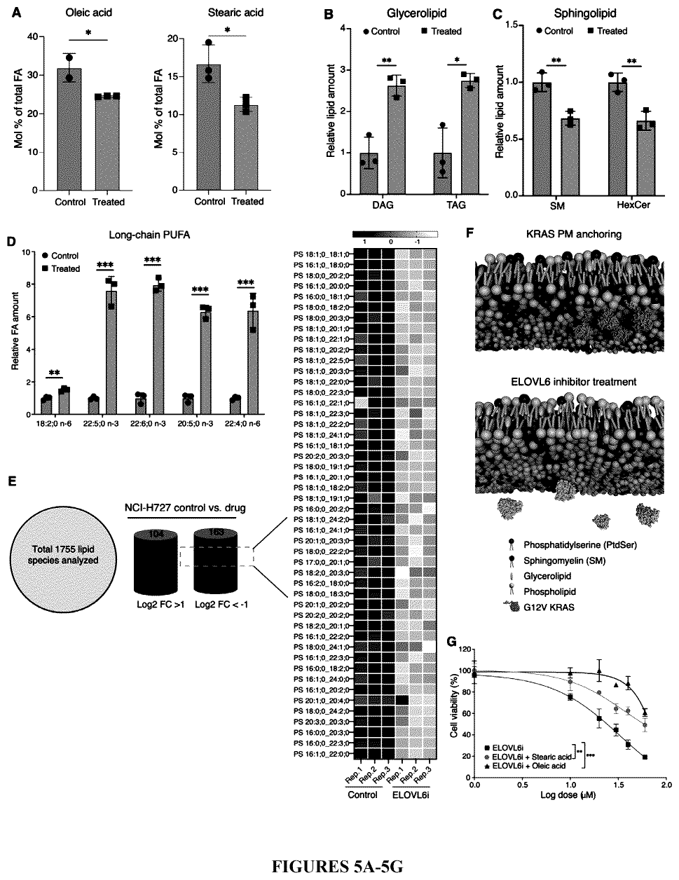

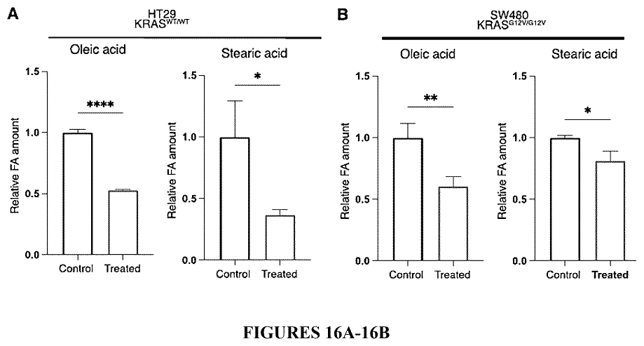

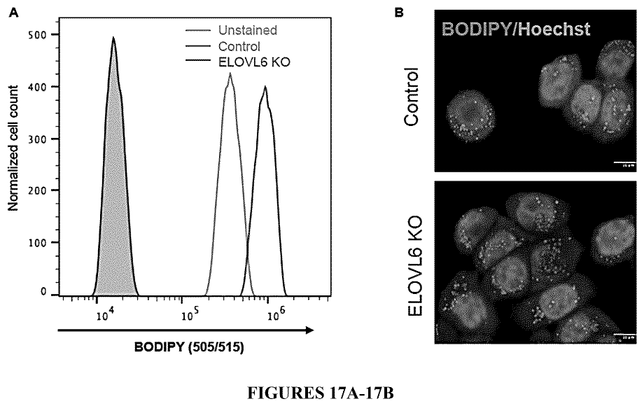

ELOVL6 plays an important role in de novo synthesis of long-chain fatty acids (20), catalyzing the sequential elongation of palmitate to stearate and subsequently to oleate in conjunction with stearoyl-CoA desaturase 1 (SCD-1) (15). ELOVL6 gene disruption mice show reduced stearate and oleate levels but are viable and fertile (15, 21). To assess the effects of ELOVL6 inhibition on depleting relevant fatty acids, Applicant conducted targeted lipidomic analysis on fatty acids in vehicle-treated controls or ELOVL6i-treated cells. A significant reduction in the levels of stearate and oleate in ELOVL6i treated cells was observed as anticipated (FIGS. 5A and 16). In addition to the alterations in fatty acid biosynthesis, Applicant also observed the accumulation of cellular droplets in ELOVL6 KO cells (FIG. 17), consistent with previous findings (22).

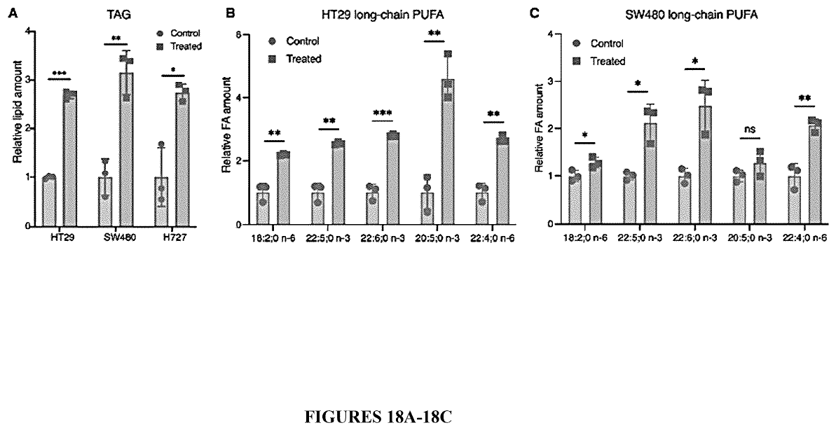

Applicant used shotgun lipidomic analysis to identify lipids that differed between the vehicle and ELOVL6i treated cells. Notable increases in glycerolipid abundance, including diacylglycerol (DAG) and triacylglycerol (TAG), were found in ELOVL6i treated cells (FIGS. 5B and 18A). A significant reduction in sphingolipids, including sphingomyelin (SM) and Hexosyl-Ceramide (HexCer), was also observed (FIG. 5C). Notably, levels of PtdSer, an anionic phospholipid primarily concentrated in the inner leaflet in the PM (23) (see FIG. 5F), was significantly altered in ELOVL6i treated cells (FIG. 5E). Lipidomic analysis of ELOVL6i treated cells demonstrated significant alterations in PM lipid abundances (FIGS. 5E). Without wishing to be bound by any particular theory, given the prior studies indicating that PM mislocalization of G12V KRAS can be caused by reduced PtdSer levels and depletion of asymmetric forms of this lipid specifically (24-27), it is likely that loss of G12V KRAS membrane localization and subsequent degradation is triggered by changes in plasma membrane lipid composition (FIG. 5F).

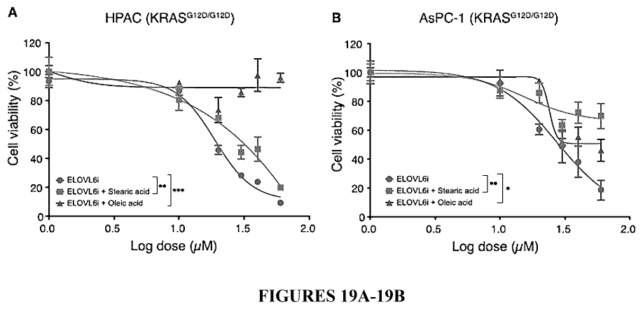

To explore whether the phenotype induced by ELOVL6 inhibition could be rescued, Applicant added exogenous lipid supplements including oleic acid or stearic acid into the cell medium during ELOVL6i treatment. This supplementation enhanced cell viability in G12V or G12D KRAS cell lines (FIGS. 5G, and 19) and provides a further link between ELOVL6 activity and cellular viability linked to oncogenic KRAS variants.

### Discussion

The urgent need for mutation-specific therapeutic intervention in G12V KRAS tumors prompted investigation into allele-specific modulators. The approach described herein, integrating genome-wide CRISPR/Cas9 based KO screening and high-throughput microfluidic cell sorting, identified ELOVL6 as a selective modulator of G12V KRAS protein levels.

ELOVL6 is a key enzyme in the elongation of 16-carbon saturated and monounsaturated fatty acids to 18-carbon fatty acids, which is the rate-limiting step for the de novo synthesis of VLCFs (20). Previous studies predominantly focused on the role of ELOVL6 activity in insulin resistance (15, 20, 21, 28, 29) with ELOVL6 deficient mice exhibiting amelioration of insulin resistance (15) and high ELOVL6 expression correlating with severity of hepatosteatosis in nonalcoholic steatohepatitis (30).

The methods utilized herein uncovered a new link between ELOVL6 activity and G12V KRAS protein levels. ELOVL6 KO cells exhibited selective reduction in G12V KRAS protein levels without significant alteration of WT KRAS protein levels. Functional analysis further demonstrated a link between ELOVL6 and G12V KRAS-driven cancer cell proliferation. Applicant also observed a significant reduction in ERK phosphorylation, mitigating aberrant KRAS signaling.

Investigation into ELOVL6 inhibition and GTP-bound KRAS, a hallmark of KRAS activation, demonstrated a marked reduction in active KRAS across multiple cell lines expressing G12V or G12D KRAS as well as other mutants. The antiproliferative effect of ELOVL6 inhibitor treatment varied across a panel of human KRAS mutant cell lines, in agreement with previous studies indicating that KRAS mutant tumors exhibit different levels of KRAS dependency (31). Pancreatic cancer cell lines were especially sensitive. This could partly be due to overexpression of lipogenic enzymes as activated lipid synthesis is more pronounced in pancreatic cancer patients (32). Importantly, Applicant's investigation extended to in vivo anti-tumor efficacy of the ELOVL6i in G12V KRAS mutant xenograft models. Reduction in tumor growth rate and suppression of oncogenic signaling pathways supports targeting ELOVL6 in G12V KRAS-driven tumors.

The cellular lipid composition, modulated by ELOVL6 activity, emerged as a critical regulator of mutant KRAS. ELOVL6 inhibition hindered G12V KRAS localization at the cell membrane, as determined by live cell imaging. Lipidomic analysis showed alterations in lipid species linked to G12V KRAS PM anchoring. Applicant observed increases in long-chain PUFA in ELOVL6i treated cells. The formation of active KRAS nanoclusters at the plasma membrane is critical for the signaling function of the protein (33), and ELOVL6 inhibition appears to deplete critical membrane components derived from PUFAs. Furthermore, a significant decrease in PtdSer abundance is especially noteworthy. The capability of G12V KRAS anchor to selectively bind distinct subclasses of PtdSer, with a preference for assembling asymmetric PtdSer is crucial for the structural integrity of KRAS nanoclusters (27). Specifically, G12V KRAS does not engage with fully saturated PtdSer, however, mono- and di-unsaturated PtdSer species can facilitate G12V KRAS PM binding (27). A major effector of KRAS, RAF, possesses separate domains for binding PtdSer (34-36). The binding of PS, in addition to the binding of the GTP-bound active KRAS, is required for the proper activation and kinase activity of RAF (34-36). Therefore, the lipid composition is crucial for mutant KRAS-driven aberrant signaling. Reduction in these critical PtdSer species was observed upon ELOVL6i treatment.

ELOVL6 inhibition induced alterations in lipid composition that may impact the structural integrity of G12V KRAS PM anchoring and nanoclustering, affecting mutant KRAS more profoundly than WT KRAS. Lipid add-back experiments with exogenous oleic acid or stearic acid, the two major lipid products from ELOVL6 fatty acid elongation reaction, rescued cell viability, solidifying the connection between altered lipid compositions and KRAS-dependent survival. These findings strongly support the hypothesis that fatty acid elongation activity attenuation leads to selective degradation of G12V KRAS. The modulation of PM lipid composition as a means to target mutant KRAS driven cancers has been proposed previously (37) and in general, lipid metabolism in cancer is a vulnerability that could be targeted for a new generation of therapies (38).

In conclusion, the results presented herein may provide new avenues for ELOVL6-targeted therapeutic development, offering a targeted route for mutant KRAS-driven cancers. Future investigations will be crucial to validate the translational potential of targeting ELOVL6 in G12V KRAS-driven tumors and explore its broader implications in mutant KRAS oncology.

### Materials and Methods

### Cell Culture

The cell lines used are listed in Table 1. All culture media were supplemented with 10% fetal bovine serum (FBS) (Millipore Sigma, F4135) and 1% penicillin/streptomycin (Thermo Fisher Scientific, 15140163). Cells were kept at 37° C., with 5% CO2.

TABLE 1
List of cell lines used in the examples.

| Cell line | Tissue | Culture Medium | Catalog | Supplier |
|---|---|---|---|---|
| GP2D | Colon | DMEM | 95090714-1VL | Sigma |
| HCT116 | Colon | McCoy 5A | CCL-247 | ATCC |
| HT29 | Colon | McCoy 5A | HTB-38 | ATCC |
| KM12 | Colon | DMEM | NIA | NU, DTC |
| LS180 | Colon | EMEM | CL-188 | ATCC |
| LS513 | Colon | RPMI-1640 | CRL-2134 | ATCC |
| LS1034 | Colon | RPMI-1640 | CRL-2158 | ATCC |
| SK-CO-1 | Colon | EMEM | HTB-39 | ATCC |
| SW403 | Colon | DMEM | CCL-230 | ATCC |
| SW480 | Colon | DMEM | CCL-228 | ATCC |
| A-427 | Lung | EMEM | HTB-53 | ATCC |
| CALU-6 | Lung | EMEM | HTB-56 | ATCC |
| COR-L23 | Lung | RPMI-1640 | 92031919-1VL | Sigma |
| HOP92 | Lung | RPMI-1640 | NIA | NU, DTC |
| NCI-H1944 | Lung | RPMI-1640 | H1944 | ATCC |
| NCI-H2444 | Lung | RPMI-1640 | CRL-5945 | ATCC |
| NCI-H441 | Lung | RPMI-1640 | HTB-174 | ATCC |
| NCI-H647 | Lung | RPMI-1640 | H647 | ATCC |
| NCI-H727 | Lung | RPMI-1640 | CRL-5815 | ATCC |
| SK-LU-1 | Lung | EMEM | HTB-57 | ATCC |
| SW1573 | Lung | DMEM | CRL-2170 | ATCC |
| AsPC-1 | Pancreas | RPMI-1640 | CRL-1682 | ATCC |
| BxPC3 | Pancreas | RPMI-1640 | CRL-1687 | ATCC |

### Lentiviral Production and Transduction

All lentivirus was produced using lenti-X 293 T cells (Takara Bio, #632180). For the construction of the TKOv3 genome-wide library, 8.0 μg of TKOv3 pooled plasmid (Addgene, #90294) (12) was co-transfected into lenti-X 293T cells along with 4.8 μg of psPAX2 (Addgene, #12260) and 3.2 μg of pMD2.G (Addgene, #12259) in 15-cm culture plate (Sarstedt). The plasmid mixture was added to 48 μl of X-tremeGene™ 9 DNA transfection reagent (Life Tech, #31985-070) that preincubated in 750 μl of OptiMEM (Gibco, #31985062) for 5 minutes, and further incubated for 25 minutes before added dropwise to lenti-X HEK293T cells. After 18 hours, the medium was replaced with fresh DMEM. Viral particles were harvested 48 hours post-media exchange, snap-frozen in liquid nitrogen, and store at −80° C. Individual KRAS and ELOVL6 CRISPR KO lentivirus was generated following the polyethyleneimine (PEI) transfection protocol from Addgene. Specifically, sgRNAs targeting the individual gene were cloned into the lentiCRISPRv2 plasmid (Addgene, #52961). The 20 base-pair sgRNA sequences are listed in Table 2.

TABLE 2
sgRNA sequences used in the CRISPR knockouts.

| Gene | sgRNA targeting region (5′-3′) |
|---|---|
| KRAS guide1 | GGACCAGTACATGAGGACTG |
| | (SEQ ID NO: 1) |
| KRAS guide2 | TGATGGAGAAACCTGTCTCT |
| | (SEQ ID NO: 2) |
| KRAS guide3 | AAGAGGAGTACAGTGCAATG |
| | (SEQ ID NO: 3) |
| ELOVL6 guide 1 | TTATATTCGGTGGTCGGCAC |
| | (SEQ ID NO: 4) |
| ELOVL6 guide 2 | ACGAGAATGAAGCCATCCAA |
| | (SEQ ID NO: 5) |
| ELOVL6 guide 3 | TACAAAGACATGGTTGCCGG |
| | (SEQ ID NO: 6) |
| Lacz (E. coli) | CCCGAATCTCTATCGTGCGG |
| | SEQ ID NO: 7) |

Lentivirus was transduced into 3×105 cells on six-well plates with media supplemented with 8 μg/ml polybrene (Sigma, #TR-1003-G). After 24-hour transduction, medium was exchanged for fresh medium containing 2 μg/ml puromycin (Thermo Fisher Scientific, #A1113803). After 48-hours of antibiotic selection, multiplicity of infection (MOI) was determined by comparing the number of cells remained at indicated viral load with or without puromycin selection using 0.1% crystal violet (Sigma, #V5265-500 ML) staining and reading the absorbance at 590 nm.

### Genome-wide CRISPR KO Screens

CRISPR screens in SW480 and HT29 cells were performed as previously described (12). In brief, 120×106 were transduced with TKOv3 library at an MOI of 0.3. Medium was changed 24 hr post-transduction to puromycin-containing medium (2 μg/ml). After two rounds of puromycin selection, cells were split into three replicates, passaged every 3-4 days. Subsequently, cells were collected for genomic DNA extraction at T0 after puromycin selection, and at every passage. For sorting, cells were fixed with 4% paraformaldehyde after collection. Sorting was performed when the cells have gone through 12-population doubling (around day 14). The corresponding Tx unsorted sample was used as reference for the NGS-based gRNA enrichment analysis.

### Microfluidic Chip Fabrication and Operation

Cytosort chips were fabricated using 3D-printed molds as previously described (13). The microfluidic devices have two compartments that gradually increase in depth, ranges from 200 μm to 400 μm, which generates a decreasing fluidic velocity. The fluidic channel contains X-shaped microfabricated structure that form capture pockets to trap desired cells. CRISPR-edited cells were fixed with 4% paraformaldehyde (Fisher Scientific, #AA47377-9M) for 20 minutes and permeabilized with 0.2% Triton X-100 (Sigma, X100-100 ML) for 10 minutes. Cells were then labeled with biotin-conjugated KRAS antibody (Stressmarq, #SPC-777D-BI) overnight at 4° C. Next day, cells were labeled with anti-biotin microbeads (Miltenyi Biotec, #130-105-637) for 1 hour. Immunomagnetically functionalized cells then proceeded to sorting. To prepare the Cytosort chips for cell sorting, the chips were primed with sterile water containing 1% Pluronic F68 (Thermo Fisher Scientific, #24040032) under constant hydraulic pressure overnight to remove air bubbles. The chips are placed on arrays of N52 magnets (K&J magnetics, #D14-N52) before connected to a syringe pump to withdraw the fluid during sample processing. PBS was withdrawn through the chip to displace the Pluronic F68 solution before loading the cells. Solution containing the cells that was collected from zone 1, zone 2 and the syringe connected to the outlet of the chip represents high-KRAS, medium-KRAS, and low-KRAS expressing cells, respectively.

### Genomic DNA Extraction and PCR

Genomic DNA (gDNA) was extracted from cell pellets using the QIAamp DNA blood Maxi Kit (Qiagen) following the manufacturer's instructions. The gDNA extraction from the fixed cells were incubated with lysis buffer and proteinase K at 55° C. overnight, following addition of RNAse A solution the next day and processed using the QIAamp DNA Blood Maxi Kit. gDNA was sent to the Princess Margret Genomics Center (PMGC, Toronto, Canada) for two-step PCR amplification of the sgRNA regions and barcoded. The primers used for sgRNA region amplification are listed in Table 3. NGS was done using Illumina NovaSeq 6000 with at least 1 million reads per sample.

TABLE 3
Primers used in sgRNA region amplification
and quantitative PCR

| | Forward (5′-3′) | Reverse (5′-3′) |
|---|---|---|
| NGS | | |
| sgRNA region | GAGGGCCTATTTCCCATGATTC | GTTGCGAAAAAGAACGTTCACG |
| | (SEQ ID NO: 8) | G (SEQ ID NO: 9) |
| RT-qPCR | | |
| ELOVL6 set1 | CTGGAAGAAATCTTTCCTGTTT | ACTAATGGCTTCCTCAGTTC |
| | (SEQ ID NO: 10) | (SEQ ID NO: 11) |
| ELOVL6 set2 | CTGCTCTGTATGCTGCCTTTAT | GGTCAGAGACCAGAGCACTAA |
| | (SEQ ID NO: 12) | (SEQ ID NO: 13) |
| KRAS set1 | TGGACGAATATGATCCAACAAT | ATTGCACTGTACTCCTCTTGAC |
| | AGA (SEQ ID NO: 14) | (SEQ ID NO: 15) |
| KRAS set2 | GGAGGGCTTTCTTTGTGTATTTG | GGTACATCTTCAGAGTCCTTAA |
| | (SEQ ID NO: 16) | CTC (SEQ ID NO: 17) |
| GAPDH | GAAGGTGAAGGTCGGAGT | GAAGATGGTGATGGGATTTC |
| | (SEQ ID NO: 18) | (SEQ ID NO: 19) |
| TIDE | | |
| KRAS set 1 | CTTAAGCGTCGATGGAGGAG | CTTGAAACCCAAGGTACATTTC |
| | (SEQ ID NO: 20) | AG (SEQ ID NO: 21) |
| KRAS set 2 | CGATACACGTCTGCAGTCAA | CCCTCTCACGAAACTCTGAAAT |
| | (SEQ ID NO: 22) | A (SEQ ID NO: 23) |
| ELOVL6 set 1 | GACAGGAGAACACTCGAAATCA | CACCCTGTGCACACACATA |
| | (SEQ ID NO: 24) | (SEQ ID NO: 25) |
| ELOVL6 set 2 | GCGCAGGCAATGCTCAG | ACAGACCTCGCGCTTCA |
| | (SEQ ID NO: 26) | (SEQ ID NO: 27) |

NGS Data and sgRNA Enrichment Analyses

The NGS data were mapped and raw sgRNA counts were converted to reads per million (rpm) by PMGC. For sgRNA enrichment analysis and fold change were obtained using MAGeCK pipeline (14) by setting comparisons between the unsorted and the sorted/enriched samples. To identify the top candidate genes (FDR<0.3) from the genome-wide screens, paired analysis was conducted across 3 replicates for each cell line. Pathway enrichment analysis was performed using Metascape (37).

### RNA Extraction and Real-time Quantitative PCR

Total RNA was extracted using the PureLink RNA mini kit (Thermo Fisher Scientific, #12183018A), followed by conversion to cDNA using the superscript VILO cDNA synthesis kit (Thermo Fisher Scientific, #11754050). Samples are prepared in triplicates in 384-well plate with SYBR green qPCR master mix (Thermo Fisher Scientific, #4367659). Subsequently, qPCR was run on a QuantStudio 6 Flex Real-Time PCR system (Applied Biosystems). The sequences of all primers are listed in Table 3 (above).

### Flow Cytometry

To label intracellular markers, live cells were fixed with 4% paraformaldehyde (Fisher Scientific, #AA47377-9M) for 20 minutes, washed with PBS, permeabilized with 0.2% Triton X-100 (Sigma, X100-100ML) for 10 minutes, blocked with PBS with 2% BSA (flow buffer) for 30 minutes then stained with the antibodies prepared in flow buffer. Cells were incubated with primary antibodies for 1-hour at room temperature. Primary antibodies used were: anti-KRAS (Proteintech, #12063-1-AP), anti-phospho-ERK (Thr202/Tyr204) (Cell Signaling Technology, #4370S), and anti-ERK (Cell Signaling Technology, #4695S). After wash with flow buffer, cells were incubated with secondary antibodies for 30 minutes. Secondary antibodies used were anti-rabbit Alexa-488 (Thermo Fisher Scientific, #A32731), anti-rabbit APC (Thermo Fisher Scientific, #A-10931), and anti-rabbit PE (Cell Signaling Technology, #79408S). For lipid accumulation, cells were stained with BODIPY 505/515 (Fisher Scientific, #D3921). Stained samples and the isotype control were analyzed immediately by flow cytometers. Data were analyzed using the FlowJo software. MFI was defined as median fluorescence across the cell population.

### Immunoblotting

Whole-cell extracts were prepared using RIPA buffer (Sigma, #R0278) supplemented with protease and phosphatase inhibitor cocktail (Thermo Fisher Scientific, #78442) on ice for 20 minutes. Subsequently, the lysates were centrifuged at 10,000 g for 20 minutes, and the protein concentration was determined using the BCA assay (Thermo Fisher Scientific, #A55864). The proteins were separated on 4-15% precast protein gels (Bio-Rad, #4568084) in 1×-Tris-Glycine-SDS buffer (Bio-Rad, #1610772) at a constant voltage of 100V and transferred onto nitrocellulose membrane (Bio-Rad, #1620115) using 1× Tris-Glycine Buffer (Bio-Rad, 1610734) at 100V for 1 hour. Following transfer, membrane was blocked in 5% skim milk for 1 hour, then incubated at 4° C. with primary antibodies. Detection was performed using horseradish peroxidase (HRP)-conjugated secondary antibodies. Antibodies used for western blotting are listed in Table 4. Blots were visualized with chemiluminescent substrate (Thermo Fisher Scientific, #34580) using Azure imaging system. Immunoblots were quantified using ImageJ.

TABLE 4
Antibodies used in western blotting.

| | | | | | Research |
|---|---|---|---|---|---|
| | | | | | Resource |
| Antibody | Catalog | Supplier | Dilution | Host | Identifier |
| KRAS | 703345 | Thermo | 1:1000 | Rabbit | AB_2784575 |
| | | Fisher | | | |
| G12V KRAS | 14412S | CST | 1:500 | Rabbit | AB_2714031 |
| ERK | 4695s | CST | 1:1000 | Rabbit | AB_390779 |
| p-ERK (T202/Y204) | 4370s | CST | 1:1000 | Rabbit | AB_2315112 |
| AKT | 9272s | CST | 1:1000 | Rabbit | AB_329827 |
| p-AKT (Ser473) | 4060s | CST | 1:500 | Rabbit | AB_2315049 |
| Beta-tubulin | 2146s | CST | 1:1000 | Rabbit | AB_2210545 |
| GAPDH | MA5-15738 | Thermo | 1:2000 | Mouse | AB_10977387 |
| | | Fisher | | | |
| Histone H3 | 4499s | CST | 1:2000 | Rabbit | AB_10544537 |
| Beta-actin | MA5-15739 | Thermo | 1:2000 | Mouse | AB_10979409 |
| | | Fisher | | | |
| Goat anti-mouse HRP | 62-6520 | Thermo | 1:10000 | Goat | AB_2533947 |
| | | Fisher | | | |
| Goat anti-rabbit HRP | 31460 | Thermo | 1:10000 | Goat | AB_228341 |
| | | Fisher | | | |

### Cell Proliferation Assay

For the MTT proliferation assay (Abcam, ab322091), cells were seeded at a density of 5,000-10,000 cells/well and cultured for 1 week. MTT reagent was added to cell culture media and incubated at 37° C. for 3 hours. Subsequently, MTT solvent was added and placed on an orbital sharker in dark for 15 minutes, followed with absorbance reading at 590 nm. For the colony formation assay, 500 cells were seeded in 6-well plate and cultured for 2-3 weeks. Cells were washed with PBS, fixed with ice-cold methanol, and stained with 0.1% crystal violet and absorbance reading at 590 nm. Cell proliferation difference was determined using the unmodified cells as the baseline control.

### RAS Activation Assay

Cells were treated with 40 μM of ELOVL6 inhibitor ELOVL6-IN-2 (MCE, HY-12146) for 3days and collected for protein extraction. RAS activity was assessed utilizing the active Ras pull-down and detection kit (Thermo Fisher Scientific, #16117). In brief, GST-RAF1 RBD and glutathione agarose resin were combined with whole-cell lysates and incubated on a rotator for 1 hour at 4° C. This was followed by three washes and elution with 2× SDS-PAGE loading buffer. The eluted samples were then subjected to western blotting to detect GTP-RAS.

### Immunofluorescence Microscopy

HT29 and SW480 cells were transduced with mCherry-WT-KRAS (Addgene, #153335) or mCherry-G12V-KRAS (Addgene, #153336) plasmid for stable mCherry-KRAS expression. Cells were seeded onto 35 mm glass bottom dish (Fisher Scientific, #NC0794151). ELOVL6 inhibitor was added at 40 μM prior to imaging. Cells were stained with CellBrite™ Steady 488 membrane dye (Biotium, #30106 T) for 30 minutes. Live cells were imaged using Nikon W1 dual cam spinning disk confocal with TIRF and a 100× objective. Five random fields of view (FOV) were selected, and an image/FOV was taken every 15 minutes for a duration of 20 hours. Images were analyzed by Fiji Image J. The mean fluorescence intensity of mCherry-KRAS was normalized to the time zero measurement of each FOV to generate relative mCherry-KRAS expression across the experiment's timespan. The Pearson's coefficient plugin JACop from ImageJ was used for quantification co-localization between mCherry-KRAS and cell membrane dye.

### Xenograft Studies

To establish cell-line-derived xenograft models, 5 million cells (SW403 and NCI-H441) suspended in PBS were subcutaneously injected to the right flanks of 8-week-old female athymic nude (nu/nu) mice purchased from the Jackson Laboratory. Once tumor reached approximately 50 mm3 volume, mice were randomized into groups (n=10) and treated with vehicle control (corn oil) or ELOVL6 inhibitor (dissolved in corn oil) dosed at 300 mg/kg. Treatment was administered twice daily by oral gavage. Tumor growth was measured 2-3 times per week using a caliper. Tumors were collected at the indicated timepoint and at the endpoint for analysis. For KRAS downstream signaling analysis, tumors were dissociated into single cells for flow cytometry analysis and protein was extract for western blotting.

### Immunohistochemical Staining

Tumor specimens were fixed in 10% formalin for at least 24 hours. Then washed with PBS and stored in 70% ethanol until paraffin embedding. Slides were stained using Dako Autostainer Plus. The following antibodies were used for staining: KRAS (Proteintech, #12063-1-AP), Ki-67 (Abcam, Ab15580), phospho-ERK (Cell Signaling Technology, #4370S), and ERK (Cell Signaling Technology, #4695s). Slides were then incubated with HRP labelled polymer anti-rabbit antibody (Agilent, K4003). All slides were rehydrated through xylene and alcohol, them mounted, and cover slipped. Slides were scanned at 20× using a Nano Zoomer 2.0 HT (Hamamatsu Photonics).

### Cell Viability Assay

The cells were seeded in triplicates in 96-well plates at a density of 5,000-10,000 cells per well and treated with the indicated concentrations of ELOVL6 inhibitor. Assay plates were subjected to daily media exchange with fresh ELOVL6 inhibitor. Following treatment, cell viability was assessed after a period of 1-week treatment using the CellTiter-Glo Luminescent Cell Viability Assay (Promega, #G7571) with luminescence readings taken on a Cytation™ 3 cell imaging multi-mode reader (Fisher Scientific). The background value was indicated by cell medium without cells and was subtracted from the raw data. The fold change in cell viability was determined relative to control cells that received no treatment.

For the lipid-addback experiment, cells were treated with the indicated concentrations of ELOVL6 inhibitor in 200 μM of oleic acid (Sigma, #O1383-1G) or 20 μM of stearic acid (Sigma, #S4751-1G) supplemented medium.

### Lipidomic Analysis

Cells were treated with ELOVL6 inhibitor at 40 μM for 1-week. Cell pellets were stored at −80° C. until sent for lipidomic analysis. Mass spectrometry-based shotgun untargeted lipid analysis and targeted fatty acids analysis were conducted by Lipotype (Dresden, Germany). Lipids were extracted using chloroform and methanol procedure as previously described (38). Samples were analyzed by automated direct infusion and orbitrap mass spectrometry. LipotypeXplorer was used for identification of lipids in the raw mass spectra (39). Alterations in lipid composition were determined based on mol % of total lipid normalized to untreated control samples.

### References

1. M. Malumbres, Nat. Rev. Cancer. 3, 459-465 (2003).

2. A. R. Moore, Nat. Rev. Drug. Discov. 19, 533-552 (2020).

3. M. H. Hofmann,. Cancer Discov. 12, 924-937 (2022).

4. S. R. Punekar, Nat. Rev. Clin. Oncol. 19, 637-655 (2022).

5. S. Lu, Sci. Rep. 6, 21949 (2016).

6. J. M. Ostrem, Nature 503, 548-551 (2013).

7. P. Lito, Science 351, 604-608 (2016).

8. J. Canon, Nature 575, 217-223 (2019).

9. J. Hallin, Cancer Discov. 10, 54-71 (2020).

10. D. S. Hong, N. Engl. J. Med. 383, 1207-1217 (2020).

11. F. Skoulidis, N. Engl. J. Med. 384, 2371-2381 (2021).

12. T. Hart, G3 (Bethesda) 7, 2719-2727 (2017).

13. Z. Wang, Nat. Biomed. Eng. 6, 108-117 (2022).

14. W. Li, Genome Biol. 15, 554 (2014).

15. T. Matsuzaka, Nat. Med. 13, 1193-1202 (2007).

16. L. Kyselova, Prog. Lipid. Res. 87, 101180 (2022).

17. T. Nagase, J. Med. Chem. 52, 4111-4114 (2009).

18. K. Shimamura, J. Pharmacol. Exp. Ther. 330, 249-256 (2009).

19. K. Shimamura, Eur. J. Pharmacol. 630, 34-41 (2010).

20. T. Matsuzaka, J. Mol. Med (Berl). 87, 379-384 (2009).

21. Y. A. Moon, J. Lipid. Res. 55, 2597-2605 (2014).

22. Y. C. Su, Sci. Rep. 8, 6586 (2018).

23. P. A. Leventis, Annu Rev Biophys 39, 407-427 (2010).

24. Y. Zhou, Mol. Cell. Biol. 34, 862-876 (2014).

25. Y. Zhou, Proc. Natl. Acad. Sci. U.S.A. 118, (2021).

26. Y. Zhou, Science 349, 873-876 (2015).

27. Y. Zhou, Cell 168, 239-251 e216 (2017).

28. H. Shimano, Prog. Lipid. Res. 51, 267-271 (2012).

29. H. Zhao, Diabetes 66, 1833-1846 (2017).

30. T. Matsuzaka, Hepatology 56, 2199-2208 (2012).

31. A. Singh, Cancer Cell 39, 441-442 (2021).

32. Y. Sunami, Cancers (Basel) 10, (2017).

33. N. R. Fuentes, Cancer Res. 78, 3899-3912 (2018).

34. S. Ghosh, J. Biol. Chem. 271, 8472-8480 (1996).

35. Z. L. Li, ACS Cent. Sci. 4, 298-305 (2018).

36. M. R. Packer, Proc. Natl. Acad. Sci. U.S.A. 118, (2021).

37. W. E. Kattan, Proc. Natl. Acad. Sci. U. S. A. 118, (2021).

38. N. Koundouros, Br. J. Cancer. 122, 4-22 (2020).

39. Y. Zhou, Nat. Commun. 10, 1523 (2019).

40. M. A. Surma, Sci. Rep. 11, 19364 (2021).

41. R. Herzog, Genome Biol. 12, R8 (2011).

### Additional Embodiments

1. A method to identify genetic modulators genes of KRAS protein expression in a KRAS mutant variant comprising:
a) obtaining genome-wide CRISPR edited cells for cancer cell line populations expressing either wild type KRAS or a KRAS mutant variant;
b) using microfluidic cell sorting to sort cells in each population into endogenous KRAS protein levels based subpopulations;
c) selecting low KRAS-expressing cells; and
d) identifying candidate genetic modulator genes in the low KRAS-expressing cells;
wherein the low KRAS-expressing cells represent about the bottom 1-25% of KRAS-expressors in each population,
wherein the candidate genetic modulators genes are enriched in the KRAS mutant variant and not enriched in the wild type KRAS and,
wherein the candidate genetic modulator gene encodes a protein associated with KRAS functional regulation and/or production in the KRAS mutant variant but not in the wild-type KRAS cell population.

2. The method of embodiment 1, wherein the KRAS mutant is selected from G12C, G12R, G13D, Q61X, A146T, G12V and G12D.

3. The method of embodiment 1, wherein the Toronto Knockout V3 guide library is used for the genome-wide CRISPR screen.

4. The method of any of embodiments 1-3, wherein the KRAS mutant variant cell population is from a colon cancer, a lung cancer, or a pancreatic cancer sample.

5. The method of any of embodiments 1-3, wherein the KRAS mutant variant cell population is a cell line selected from SW1573, Mia-paca2, Capan-2, Panc 02.03, HCT116, SW480, PSN-1, Panc 03.27, SW403, NCI-H441, GP2D, LS180, COR-L23, LS1034, Capan-1, SK-LU-1, SW1990, NCI-H1944, LS513, Panc 04.03, NCI-H647, NCI-H2444, Calu-6, A427, HUP-T4, CFPAC-1, SK-CO-1, Aspc-1, NCI-H727, and HPAC.

6. A genetic modulator identified by the method of any of embodiments 1-5, wherein the genetic modulator is identified as an allele-selective KRAS modulator, wherein the genetic modulator is associated with controlling KRAS levels in at least one KRAS mutant variant.

7. The genetic modulator of embodiment 6, wherein the genetic modulator encodes an enzyme, optionally wherein the genetic modulator is ELOVL6.

8. The genetic modulator of embodiment 6, wherein the KRAS mutant variant is selected from G12C, G12R, G13D, Q61X, A146T, G12V and G12D.

9. The genetic modulator of any of embodiments 6-8, wherein the genetic modulator is ELOVL6, NCAPG, SLC9B2, TGM6, SDE2, PHB2, or PGD.

10. A method of treatment of cancer in a subject in need thereof comprising:
a) determining the KRAS mutant variant in the subject in need;
b) applying an inhibitor of the genetic modulator of any of claims 6-8,
wherein the cancer is dependent or addicted on KRAS, and wherein the inhibitor reduces KRAS levels by the subject in need, thereby inhibiting the cancer, optionally wherein the inhibitor is applied orally.

11. The method of embodiment 10, wherein the KRAS mutant variant is selected from is selected from G12C, G12R, G13D, Q61X, A146T, G12V and G12D.

12. The method of embodiment 10 or 11, wherein the cancer is colon cancer, lung cancer, or pancreatic cancer.

13. The method of any of embodiments 10-12, wherein the genetic modulator is ELOVL6, NCAPG, SLC9B2, TGM6, SDE2, PHB2, or PGD.

14. The method of embodiment 13, wherein the genetic modulator is ELOVL6.

15. The method of embodiment 14, wherein the inhibitor has the chemical structure according to Formula (I):

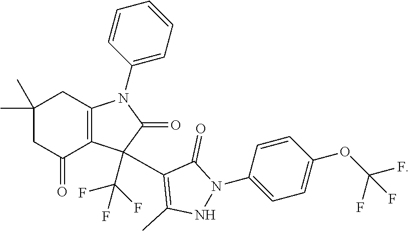 [SMILES: Cc1[nH]n(-c2ccc(OC(F)(F)F)cc2)c(=O)c1C1(C(F)(F)F)C(=O)N(c2ccccc2)C2=C1C(=O)CC(C)(C)C2]

16. A method of inhibiting the growth of cancer cells comprising:
a) determining the KRAS mutant variant in the cell population
   b) contacting the cells with an inhibitor of the genetic modulator of any of embodiments 6-8,
wherein the cancer cells are dependent or addicted on continued KRAS expression for cell survival, and wherein the inhibitor reduces KRAS levels in the cells, thereby inhibiting the growth of the cells.

17. The method of embodiment 15, wherein the cells are contacted in vitro, in situ, ex vivo, or in vivo.

18. The method of embodiment 16 or 17, wherein the KRAS mutant variant is selected from G12C, G12R, G13D, Q61X, A146T, G12V and G12D.

### EQUIVALENTS

Unless otherwise defined, all technical and scientific terms used herein have the same meaning as commonly understood by one of ordinary skill in the art to which this technology belongs.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, GenBank citations, ATCC citations, and other references mentioned herein are expressly incorporated by reference in their entirety, to the same extent as if each were incorporated by reference individually. In case of conflict, the specification, including definitions, will control.

What is claimed is:

1. A method of inhibiting growth of cancerous cells, the method comprising contacting the cancerous cells with a very long chain fatty acid elongase 6 inhibitor (ELOVL6i), wherein the cancerous cells are KRAS dependent, wherein the ELOVL6i reduces a level of KRAS expressed by the cancerous cells, relative to a level thereof prior to contacting of the ELOVL6i, thereby inhibiting the growth of the cancerous cells.

2. The method of claim 1, wherein the KRAS comprises a variant KRAS having at least one substitution relative to a wildtype human KRAS allele.

3. The method of claim 2, wherein the at least one substitution of the variant KRAS is at a position selected from G10, G12, G13, V14, L19, Q22, D33, A59, G60, Q61, R68, H95, Y96, K117 and A146, relative to the wildtype human KRAS allele.

4. The method of claim 2, wherein the at least one substitution of the variant KRAS is selected from G12C, G12R, G13D, Q61X, R68S, H95X, Y96C, A146T, G12V and G12D, relative to the wildtype human KRAS allele.

5. The method of claim 2, wherein the contacting results in a selective reduction of the level of the variant KRAS, relative to a level of the wildtype human KRAS allele.

6. The method of claim 1, wherein the contacting results in a reduction of a level of KRAS inside the cancerous cell, as compared to a level inside the cancerous cell prior to the contacting.

7. The method of claim 1, wherein the ELOVL6i is present in a drug composition.

8. The method of claim 7, wherein the drug composition is formulated for oral delivery.

9. A method of treating cancer that expresses a variant KRAS having at least one substitution relative to a wildtype human KRAS allele in a subject in need thereof, the method comprises administering to the subject a drug composition that comprises a very long chain fatty acid elongase 6 inhibitor (ELOVL6i), wherein the administering reduces a level of the variant KRAS in the subject, relative to a level of the variant KRAS prior to the administering, thereby treating the cancer that expresses the variant KRAS in the subject.

10. The method of claim 9, further comprising detecting a presence of the variant KRAS in the subject prior to the administering.

11. The method of claim 9, wherein the at least one substitution of the variant KRAS is at a position selected from G10, G12, G13, V14, L19, Q22, D33, A59, G60, Q61, R68, H95, Y96, K117 and A146, relative to the wildtype human KRAS allele.

12. The method of claim 9, wherein the at least one substitution of the variant KRAS is selected from G12C, G12R, G13D, Q61X, R68S, H95X, Y96C, A146T, G12V and G12D, relative to the wildtype human KRAS allele.

13. The method of claim 9, wherein the at least one substitution of the variant KRAS is a G12V or G12D substitution, relative to the wildtype human KRAS allele.

14. The method of claim 9, wherein the administering results in a selective reduction of the level of the variant KRAS in the subject, relative to a level of the wildtype human KRAS allele.

15. The method of claim 9, wherein the administering results in a reduced level of ERK phosphorylation and ki-67, relative to a level of ERK phosphorylation and ki-67 prior to the administering.

16. The method of claim 9, wherein the administering results in a reduced level of AKT phosphorylation, relative to a level thereof prior to the administering.

17. The method of claim 9, wherein the administering results in a reduced level of GTP-bound KRAS, relative to a level of GTP-bound KRAS prior to the administering.

18. The method of claim 9, wherein the administering results in a reduced level of very long chain fatty acids in a plasma membrane of a cancer cell of the cancer that expresses the variant KRAS, relative to a level of very long chain fatty acids in the plasma membrane of the cancer cell of cancer that expresses the variant KRAS prior to the administering, thereby reducing anchoring of KRAS to the plasma membrane of the cancer cell of the cancer that expresses the variant KRAS.

19. The method of claim 9, wherein the ELOVL6i is a small molecule having less than 1000 Da molecular weight.

20. The method of claim 9, wherein the administering is oral.

21. The method of claim 9, wherein the method reduces an expression level of a very long chain fatty acid elongase 6 (ELOVL6) relative to a level thereof prior to the administering of ELOVL6i.

22. The method of claim 9, wherein the ELOVL6i is a short interfering RNA (siRNA) or short hairpin RNA (shRNA).

23. The method of claim 9, wherein the cancer is c-MYC dependent.

24. The method of claim 9, wherein the cancer that expresses the variant KRAS is a solid tumor cancer.

25. The method of claim 9, wherein the cancer that expresses the variant KRAS is colon cancer, lung cancer, skin cancer, bile duct cancer, uterine endometrial carcinoma, testicular germ cell cancer, cervical squamous cell carcinoma, multiple myeloma or pancreatic cancer.


SEQUENCE LISTING (32 sequences)

SEQ ID NO: 1 - DNA, 20 bp, synthetic construct
  source 1..20: mol_type = other DNA
ggaccagtacatgaggactg

SEQ ID NO: 2 - DNA, 20 bp, synthetic construct
  source 1..20: mol_type = other DNA
tgatggagaaacctgtctct

SEQ ID NO: 3 - DNA, 20 bp, synthetic construct
  source 1..20: mol_type = other DNA
aagaggagtacagtgcaatg

SEQ ID NO: 4 - DNA, 20 bp, synthetic construct
  source 1..20: mol_type = other DNA
ttatattcggtggtcggcac

SEQ ID NO: 5 - DNA, 20 bp, synthetic construct
  source 1..20: mol_type = other DNA
acgagaatgaagccatccaa

SEQ ID NO: 6 - DNA, 20 bp, synthetic construct
  source 1..20: mol_type = other DNA
tacaaagacatggttgccgg

SEQ ID NO: 7 - DNA, 20 bp, synthetic construct
  source 1..20: mol_type = other DNA
cccgaatctctatcgtgcgg

SEQ ID NO: 8 - DNA, 22 bp, synthetic construct
  source 1..22: mol_type = other DNA
gagggcctatttcccatgattc

SEQ ID NO: 9 - DNA, 23 bp, synthetic construct
  source 1..23: mol_type = other DNA
gttgcgaaaaagaacgttcacgg

SEQ ID NO: 10 - DNA, 22 bp, synthetic construct
  source 1..22: mol_type = other DNA
ctggaagaaatctttcctgttt

SEQ ID NO: 11 - DNA, 20 bp, synthetic construct
  source 1..20: mol_type = other DNA
actaatggcttcctcagttc

SEQ ID NO: 12 - DNA, 22 bp, synthetic construct
  source 1..22: mol_type = other DNA
ctgctctgtatgctgcctttat

SEQ ID NO: 13 - DNA, 21 bp, synthetic construct
  source 1..21: mol_type = other DNA
ggtcagagaccagagcactaa

SEQ ID NO: 14 - DNA, 25 bp, synthetic construct
  source 1..25: mol_type = other DNA
tggacgaatatgatccaacaataga

SEQ ID NO: 15 - DNA, 22 bp, synthetic construct
  source 1..22: mol_type = other DNA
attgcactgtactcctcttgac

SEQ ID NO: 16 - DNA, 23 bp, synthetic construct
  source 1..23: mol_type = other DNA
ggagggctttctttgtgtatttg

SEQ ID NO: 17 - DNA, 25 bp, synthetic construct
  source 1..25: mol_type = other DNA
ggtacatcttcagagtccttaactc

SEQ ID NO: 18 - DNA, 18 bp, synthetic construct
  source 1..18: mol_type = other DNA
gaaggtgaaggtcggagt

SEQ ID NO: 19 - DNA, 20 bp, synthetic construct
  source 1..20: mol_type = other DNA
gaagatggtgatgggatttc

SEQ ID NO: 20 - DNA, 20 bp, synthetic construct
  source 1..20: mol_type = other DNA
cttaagcgtcgatggaggag

SEQ ID NO: 21 - DNA, 24 bp, synthetic construct
  source 1..24: mol_type = other DNA
cttgaaacccaaggtacatttcag

SEQ ID NO: 22 - DNA, 20 bp, synthetic construct
  source 1..20: mol_type = other DNA
cgatacacgtctgcagtcaa

SEQ ID NO: 23 - DNA, 23 bp, synthetic construct
  source 1..23: mol_type = other DNA
ccctctcacgaaactctgaaata

SEQ ID NO: 24 - DNA, 22 bp, synthetic construct
  source 1..22: mol_type = other DNA
gacaggagaacactcgaaatca

SEQ ID NO: 25 - DNA, 19 bp, synthetic construct
  source 1..19: mol_type = other DNA
caccctgtgcacacacata

SEQ ID NO: 26 - DNA, 17 bp, synthetic construct
  source 1..17: mol_type = other DNA
gcgcaggcaatgctcag

SEQ ID NO: 27 - DNA, 17 bp, synthetic construct
  source 1..17: mol_type = other DNA
acagacctcgcgcttca

SEQ ID NO: 28 - DNA, 17 bp, synthetic construct
  source 1..17: mol_type = other DNA
actgcctggaagttacg

SEQ ID NO: 29 - DNA, 17 bp, synthetic construct
  source 1..17: mol_type = other DNA
actgcctggaagttagg

SEQ ID NO: 30 - DNA, 17 bp, synthetic construct
  source 1..17: mol_type = other DNA
cgatgatcgttaacgga

SEQ ID NO: 31 - DNA, 17 bp, synthetic construct
  source 1..17: mol_type = other DNA
aagctgacgtcgtcagc

SEQ ID NO: 32 - DNA, 19 bp, synthetic construct
  source 1..19: mol_type = other DNA
gttaaccgtactactagat